(19) United States
(12) Patent Application Publication
(10) Pub. No.: US 2007/0049608 A1
(43) Pub. Date: Mar. 1, 2007

(54) Pyrrolopyridine, pyrrolopyrimidine and pyrazolopyridine compounds, compositions comprising them, and methods of their use

(76) Inventors: Cynthia Anne Fink, Lebanon, NJ (US); Joseph Barbosa, Lambertville, NJ (US); G. Gregory Zipp, Robbinsville, NJ (US); Jiancheng Wang, Dallas, TX (US)

Correspondence Address: LEXICON GENETICS INCORPORATED, 8800 TECHNOLOGY FOREST PLACE, THE WOODLANDS, TX, 77381-1160, US

(21) Appl. No.: 11/508,116

(22) Filed: Aug. 22, 2006

Related U.S. Application Data
(60) Provisional application No. 60/711,404, filed on Aug. 24, 2005.

(51) Int. Cl. A61K 31/519; C07D 487/02

(52) U.S. Cl. 514/265.1; 544/280

(57) ABSTRACT

Disclosed are pyrrolopyridine, pyrrolopyrimidine and pyrazolopyridine compounds, compositions comprising them, and methods of their use.

This application claims priority to U.S. provisional patent application no. 60/711,404, filed Aug. 24, 2005, the entirety of which is incorporated herein by reference.

### 1. FIELD OF THE INVENTION

This invention relates to pyrrolopyridine, pyrrolopyrimidine and pyrazolopyridine compounds, compositions comprising them, and methods of their use.

### 2. BACKGROUND OF THE INVENTION

The amino acid L-proline reportedly plays a role in regulating synaptic transmission in the mammalian brain. See, e.g., Crump et al., Molecular and Cellular Neuroscience, 13: 25-29 (1999). For example, a synaptosomal bisynthetic pathway of L-proline from omithine has been reported, and high affinity Na⁺-dependent synaptosomal uptake of L-proline has been observed. Yoneda et al., Brain Res., 239: 479-488 (1982); Balcar et al., Brain Res., 102:143-151 (1976).

In general, neurotransmitter systems typically have mechanisms that inactivate signaling, many of which work through the action of a Na⁺-dependent transporter. In this case, a Na⁺-dependent transporter for proline has been described, and the molecular entity cloned (SLC6A7 in humans). See, e.g., U.S. Pat. Nos. 5,580,775 and 5,759,788. But the transporter's specific role remains unknown. For example, the human Na⁺-dependent proline transporter is generally localized to synaptic terminals, which is consistent with a role in neurotransmitter signaling. But no high-affinity receptor has been found for proline, suggesting that it is a neuromodulator rather than a neurotransmitter. Shafqat S., et al., Molecular Pharmacology 48:219-229 (1995).

The fact that the Na⁺-dependent proline transporter is expressed in the dorsal root ganglion has led some to suggest that it may be involved in nociception, and that compounds which inhibit the transporter may be used to treat pain. See, e.g., U.S. Patent Application No. 20030152970A1. But this suggestion is not supported by experimental data.

### 3. SUMMARY OF THE INVENTION

This invention encompasses pyrrolopyridine, pyrrolopyrimidine and pyrazolopyridine compounds, pharmaceutical compositions comprising them, and methods of their use.

One embodiment of the invention encompasses a compound of formula I: 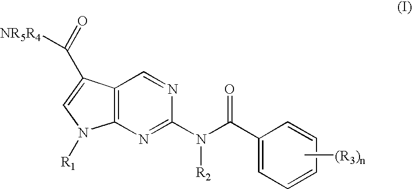
the various substituents of which are defined herein, and pharmaceutically acceptable salts and solvates thereof.

Another embodiment encompasses compounds of formula II: 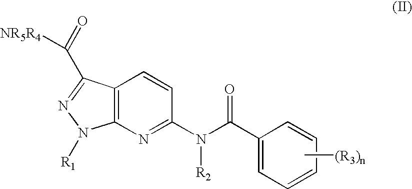
the various substituents of which are defined herein, and pharmaceutically acceptable salts and solvates thereof.

Another embodiment encompasses compounds of formula III: 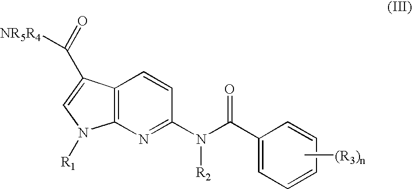
the various substituents of which are defined herein, and pharmaceutically acceptable salts and solvates thereof.

Preferred compounds inhibit the proline transporter, and particular compounds do so without substantially affecting the dopamine or glycine transporters.

Another embodiment of the invention encompasses pharmaceutical compositions of compounds of the invention (i.e., compounds disclosed herein).

Another embodiment encompasses methods of inhibiting a proline transporter, which comprise contacting the transporter with a compound of the invention.

Another embodiment encompasses methods of improving cognitive performance, and of treating, managing and/or preventing various diseases and disorders, using compounds of the invention.

### 4. DETAILED DESCRIPTION

This invention is based, in part, on the discovery that the proline transporter encoded by the human gene at map location 5q31-q32 (SLC6A7 gene; GENBANK accession no. NM_{—}014228) can be a potent modulator of mental performance in mammals. In particular, it has been found that genetically engineered mice that do not express a functional product of the murine ortholog of the SLC6A7 gene display significantly increased cognitive function, attention span, learning, and memory relative to control animals.

In view of this discovery, the protein product associated with the SLC6A7 coding region was used to discover compounds that may improve cognitive performance and may be useful in the treatment, prevention and/or management of diseases and disorders such as Alzheimer's disease, autism, cognitive disorders, dementia, learning disorders, and short- and long-term memory loss.

4.1. Definitions

Unless otherwise indicated, the term “alkenyl” means a straight chain, branched and/or cyclic hydrocarbon having from 2 to 20 (e.g., 2 to 10 or 2 to 6) carbon atoms, and including at least one carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

Unless otherwise indicated, the term “alkyl” means a straight chain, branched and/or cyclic (“cycloalkyl”) hydrocarbon having from 1 to 20 (e.g., 1 to 10 or 1 to 4) carbon atoms. Alkyl moieties having from 1 to 4 carbons are referred to as “lower alkyl.” Examples of alkyl groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). The term “alkyl” includes saturated hydrocarbons as well as alkenyl and alkynyl moieties.

Unless otherwise indicated, the term “alkylaryl” or “alkyl-aryl” means an alkyl moiety bound to an aryl moiety.

Unless otherwise indicated, the term “alkylheteroaryl” or “alkyl-heteroaryl” means an alkyl moiety bound to a heteroaryl moiety.

Unless otherwise indicated, the term “alkylheterocycle” or “alkyl-heterocycle” means an alkyl moiety bound to a heterocycle moiety.

Unless otherwise indicated, the term “alkynyl” means a straight chain, branched or cyclic hydrocarbon having from 2 to 20 (e.g., 2 to 6) carbon atoms, and including at least one carbon-carbon triple bond. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

Unless otherwise indicated, the term “alkoxy” means an —O-alkyl group. Examples of alkoxy groups include, but are not limited to, —OCH₃, —OCH₂CH₃, —O(CH₂)₂CH₃, —O(CH₂)₃CH₃, —O(CH₂)₄CH₃, and —O(CH₂)₅CH₃.

Unless otherwise indicated, the term “aryl” means an aromatic ring or an aromatic or partially aromatic ring system composed of carbon and hydrogen atoms. An aryl moiety may comprise multiple rings bound or fused together. Examples of aryl moieties include anthracenyl, azulenyl, biphenyl, fluorenyl, indan, indenyl, naphthyl, phenanthrenyl, phenyl, 1,2,3,4-tetrahydro-naphthalene, and tolyl.

Unless otherwise indicated, the term “arylalkyl” or “aryl-alkyl” means an aryl moiety bound to an alkyl moiety.

Unless otherwise indicated, the term “DTIC₅₀” means an IC₅₀ against human recombinant dopamine transporter as determined using the assay described in the Examples, below.

Unless otherwise indicated, the term “GTIC₅₀” means an IC₅₀ for human recombinant glycine transporter as determined using the assay described in the Examples, below.

Unless otherwise indicated, the terms “halogen” and “halo” encompass fluorine, chlorine, bromine, and iodine.

Unless otherwise indicated, the term “heteroalkyl” refers to an alkyl moiety (e.g., linear, branched or cyclic) in which at least one of its carbon atoms has been replaced with a heteroatom (e.g., N, O or S).

Unless otherwise indicated, the term “heteroaryl” means an aryl moiety wherein at least one of its carbon atoms has been replaced with a heteroatom (e.g., N, O or S). Examples include acridinyl, benzimidazolyl, benzofuranyl, benzoisothiazolyl, benzoisoxazolyl, benzoquinazolinyl, benzothiazolyl, benzoxazolyl, furyl, imidazolyl, indolyl, isothiazolyl, isoxazolyl, oxadiazolyl, oxazolyl, phthalazinyl, pyrazinyl, pyrazolyl, pyridazinyl, pyridyl, pyrimidinyl, pyrimidyl, pyrrolyl, quinazolinyl, quinolinyl, tetrazolyl, thiazolyl, and triazinyl.

Unless otherwise indicated, the term “heteroarylalkyl” or “heteroaryl-alkyl” means a heteroaryl moiety bound to an alkyl moiety.

Unless otherwise indicated, the term “heterocycle” refers to an aromatic, partially aromatic or non-aromatic monocyclic or polycyclic ring or ring system comprised of carbon, hydrogen and at least one heteroatom (e.g., N, O or S). A heterocycle may comprise multiple (i.e., two or more) rings fused or bound together. Heterocycles include heteroaryls. Examples include benzo[1,3]dioxolyl, 2,3-dihydro-benzo[1,4]dioxinyl, cinnolinyl, furanyl, hydantoinyl, morpholinyl, oxetanyl, oxiranyl, piperazinyl, piperidinyl, pyrrolidinonyl, pyrrolidinyl, tetrahydrofuranyl, tetrahydropyranyl, tetrahydropyridinyl, tetrahydropyrimidinyl, tetrahydrothiophenyl, tetrahydrothiopyranyl and valerolactamyl.

Unless otherwise indicated, the term “heterocyclealkyl” or “heterocycle-alkyl” refers to a heterocycle moiety bound to an alkyl moiety.

Unless otherwise indicated, the term “heterocycloalkyl” refers to a non-aromatic heterocycle.

Unless otherwise indicated, the term “heterocycloalkylalkyl” or “heterocycloalkylalkyl” refers to a heterocycloalkyl moiety bound to an alkyl moiety.

Unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

Unless otherwise indicated, the term “pharmaceutically acceptable salts” refers to salts prepared from pharmaceutically acceptable non-toxic acids or bases including inorganic acids and bases and organic acids and bases. Suitable pharmaceutically acceptable base addition salts include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art. See, e.g., Remington's Pharmaceutical Sciences (18th ed., Mack Publishing, Easton Pa.: 1990) and Remington: The Science and Practice of Pharmacy (19th ed., Mack Publishing, Easton Pa.: 1995).

Unless otherwise indicated, the terms “prevent,” “preventing” and “prevention” contemplate an action that occurs before a patient begins to suffer from the specified disease or disorder, which inhibits or reduces the severity of the disease or disorder. In other words, the terms encompass prophylaxis.

Unless otherwise indicated, a “prophylactically effective amount” of a compound is an amount sufficient to prevent a disease or condition, or one or more symptoms associated with the disease or condition, or to prevent its recurrence. A prophylactically effective amount of a compound means an amount of therapeutic agent, alone or in combination with other agents, which provides a prophylactic benefit in the prevention of the disease or condition. The term “prophylactically effective amount” can encompass an amount that improves overall prophylaxis or enhances the prophylactic efficacy of another prophylactic agent.

Unless otherwise indicated, the term “PTIC₅₀” means an IC₅₀ for human recombinant Na⁺-dependent proline transporter as determined using the assay described in the Examples, below.

Unless otherwise indicated, the term “specific proline transporter inhibitor” means a compound that has a PTIC₅₀ of less than about 200 nM.

Unless otherwise indicated, the term “substituted,” when used to describe a chemical structure or moiety, refers to a derivative of that structure or moiety wherein one or more of its hydrogen atoms is substituted with a chemical moiety or functional group such as, but not limited to, alcohol, aldehylde, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amidinyl (—C(NH)NH-alkyl or —C(NR)NH₂), amine (primary, secondary and tertiary such as alkylamino, arylamino, arylalkylamino), aroyl, aryl, aryloxy, azo, carbamoyl (—NHC(O)O-alkyl- or —OC(O)NH-alkyl), carbamyl (e.g., CONH₂, CONH-alkyl, CONH-aryl, and CONH-arylalkyl), carbonyl, carboxyl, carboxylic acid, carboxylic acid anhydride, carboxylic acid chloride, cyano, ester, epoxide, ether (e.g., methoxy, ethoxy), guanidino, halo, haloalkyl (e.g., —CCl₃, —CF₃, —C(CF₃)₃), heteroalkyl, hemiacetal, imine (primary and secondary), isocyanate, isothiocyanate, ketone, nitrile, nitro, oxo, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) and urea (—NHCONH-alkyl-).

Unless otherwise indicated, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder or one or more of its symptoms, or retards or slows the progression of the disease or disorder.

Unless otherwise indicated, the term “include” has the same meaning as “include, but are not limited to,” and the term “includes” has the same meaning as “includes, but is not limited to.” Similarly, the term “such as” has the same meaning as the term “such as, but not limited to.”

Unless otherwise indicated, one or more adjectives immediately preceding a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

It should be noted that a chemical moiety that forms part of a larger compound may be described herein using a name commonly accorded it when it exists as a single molecule or a name commonly accorded its radical. For example, the terms “pyridine” and “pyridyl” are accorded the same meaning when used to describe a moiety attached to other chemical moieties. Thus, the two phrases “XOH, wherein X is pyridyl” and “XOH, wherein X is pyridine” are accorded the same meaning, and encompass the compounds pyridin-2-ol, pyridin-3-ol and pyridin-4-ol.

It should also be noted that any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit. Structures that represent compounds with one or more chiral centers, but which do not indicate stereochemistry (e.g., with bolded or dashed lines), encompasses pure stereoisomers and mixtures (e.g., racemic mixtures) thereof. Similarly, names of compounds having one or more chiral centers that do not specify the stereochemistry of those centers encompass pure stereoisomers and mixtures thereof.

4.2. Compounds

This invention encompasses compounds of formula I: 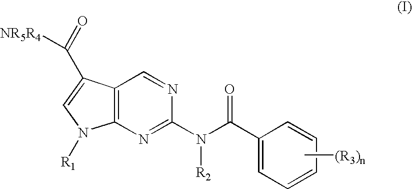
and pharmaceutically acceptable salts and solvates thereof, wherein:

R₁ is hydrogen or optionally substituted alkyl, aryl, heterocycle, alkyl-aryl or alkyl-heterocycle;

R₂ is hydrogen or optionally substituted alkyl;

each R₃ is independently halogen, amine, hydroxy, alkoxy, or optionally substituted alkyl, aryl or heterocycle;

R₄ and R₅ are each independently hydrogen or optionally substituted alkyl, aryl, heterocycle, alkyl-aryl or alkyl-heterocycle, or taken together with the nitrogen atom to which they are attached, form an optionally substituted heterocycle; and

n is 0 to 5.

In one embodiment, R₁ is optionally substituted alkyl. In another, it is alkyl (e.g., t-butyl or propyl). In another, it is optionally substituted aryl. In another, it is optionally substituted heterocycle.

In another embodiment, R₂ is hydrogen. In another, it is optionally substituted alkyl (e.g., optionally substituted methyl).

In another embodiment, R₃ is halogen. In another, it is optionally substituted alkyl (e.g., optionally substituted lower alkyl). In another, it is hydroxy.

In another embodiment, R₄ and R₅ are independently hydrogen or optionally substituted alkyl. In another, they are taken together to form optionally substituted pyridine or pyrrolidine.

In another embodiment, n is 0. In another, n is 1. In another, n is 2.

In another embodiment, R₄ and R₅ together with the nitrogen atom to which they are attached do not form 1,4-diaza-bicyclo[3.2.2]nonane. In another embodiment, R₄ and R₅ together with the nitrogen atom to which they are attached do not form piperazine-C(O)-aryl (e.g., piperazine-C(O)-phenyl).

This invention encompasses compounds of formula I-A: 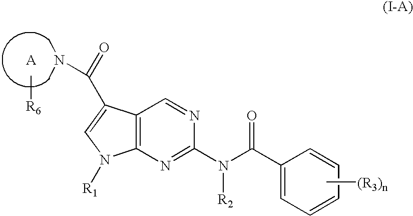
and pharmaceutically acceptable salts and solvates thereof, wherein:

A is a heterocycle;

R₁ is hydrogen or optionally substituted alkyl, aryl, heterocycle, alkyl-aryl or alkyl-heterocycle;

R₂ is hydrogen or optionally substituted alkyl;

each R₃ is independently halogen, amine, hydroxy, alkoxy, or optionally substituted alkyl, aryl or heterocycle;

R₆ is optionally substituted alkyl, aryl, heterocycle, alkyl-aryl or alkyl-heterocycle; and

n is 0 to 5.

In one embodiment, A is pyridine or pyrrolidine.

In one embodiment, R₁ is optionally substituted alkyl. In another, it is alkyl (e.g., t-butyl or propyl). In another, it is optionally substituted aryl. In another, it is optionally substituted heterocycle.

In another embodiment, R₂ is hydrogen. In another, it is optionally substituted alkyl (e.g., optionally substituted methyl).

In another embodiment, R₃ is halogen. In another, it is optionally substituted alkyl (e.g., optionally substituted lower alkyl). In another, it is hydroxy.

In another embodiment, R₆ is optionally substituted alkyl. In another, it is optionally heterocycle. In another, it is a heterocycle (e.g., pyridine or pyrrolidine).

In another embodiment, n is 0. In another, n is 1. In another, n is 2.

In another embodiment, A is not 1,4-diaza-bicyclo[3.2.2]nonane. In another embodiment, A is not piperazine-C(O)-aryl (e.g., piperazine-C(O)-phenyl).

This invention encompasses compounds of formula II: 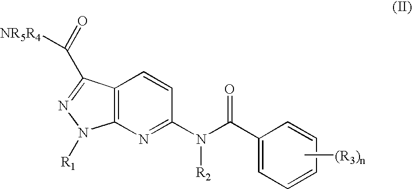
and pharmaceutically acceptable salts and solvates thereof, wherein:

R₁ is hydrogen or optionally substituted alkyl, aryl, heterocycle, alkyl-aryl or alkyl-heterocycle;

R₂ is hydrogen or optionally substituted alkyl; each R₃ is independently halogen, amine, hydroxy, alkoxy, or optionally substituted alkyl, aryl or heterocycle;

R₄ and R₅ are each independently hydrogen, or optionally substituted alkyl, aryl, heterocycle, alkyl-aryl or alkyl-heterocycle, or taken together with the nitrogen atom to which they are attached, form an optionally substituted heterocycle; and

n is 0 to 5.

In one embodiment, R₁ is optionally substituted alkyl. In another, it is alkyl (e.g., t-butyl or propyl). In another, it is optionally substituted aryl. In another, it is optionally substituted heterocycle.

In another embodiment, R₂ is hydrogen. In another, it is optionally substituted alkyl (e.g., optionally substituted methyl).

In another embodiment, R₃ is halogen. In another, it is optionally substituted alkyl (e.g., optionally substituted lower alkyl). In another, it is hydroxy.

In another embodiment, R₄ and R₅ are independently hydrogen or optionally substituted alkyl. In another, they are taken together to form optionally substituted pyridine or pyrrolidine.

In another embodiment, n is 0. In another, n is 1. In another, n is 2.

In another embodiment, R₄ and R₅ together with the nitrogen atom to which they are attached do not form 1,4-diaza-bicyclo[3.2.2]nonane. In another embodiment, R₄ and R₅ together with the nitrogen atom to which they are attached do not form piperazine-C(O)-aryl (e.g., piperazine-C(O)-phenyl).

This invention encompasses compounds of formula II-A: 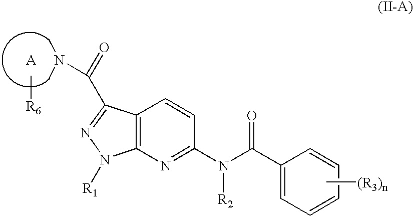
and pharmaceutically acceptable salts and solvates thereof, wherein:

A is a heterocycle;

R₁ is hydrogen or optionally substituted alkyl, aryl, heterocycle, alkyl-aryl or alkyl-heterocycle;

R₂ is hydrogen or optionally substituted alkyl;

each R₃ is independently halogen, amine, hydroxy, alkoxy, or optionally substituted alkyl, aryl or heterocycle;

R₆ is optionally substituted alkyl, aryl, heterocycle, alkyl-aryl or alkyl-heterocycle; and

n is 0 to 5.

In one embodiment, A is pyridine or pyrrolidine.

In one embodiment, R₁ is optionally substituted alkyl. In another, it is alkyl (e.g., t-butyl or propyl). In another, it is optionally substituted aryl. In another, it is optionally substituted heterocycle.

In another embodiment, R₂ is hydrogen. In another, it is optionally substituted alkyl (e.g., optionally substituted methyl).

In another embodiment, R₃ is halogen. In another, it is optionally substituted alkyl (e.g., optionally substituted lower alkyl). In another, it is hydroxy.

In another embodiment, R₆ is optionally substituted alkyl. In another, it is optionally heterocycle. In another, it is a heterocycle (e.g., pyridine or pyrrolidine).

In another embodiment, n is 0. In another, n is 1. In another, n is 2.

In another embodiment, A is not 1,4-diaza-bicyclo[3.2.2]nonane. In another embodiment, A is not piperazine-C(O)-aryl (e.g., piperazine-C(O)-phenyl).

This invention encompasses compounds of formula III: 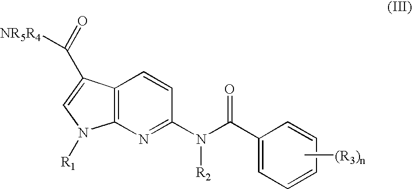
and pharmaceutically acceptable salts and solvates thereof, wherein:

R₁ is hydrogen or optionally substituted alkyl, aryl, heterocycle, alkyl-aryl or alkyl-heterocycle;

R₂ is hydrogen or optionally substituted alkyl;

each R₃ is independently halogen, amine, hydroxy, alkoxy, or optionally substituted alkyl, aryl or heterocycle;

R₄ and R₅ are each independently hydrogen, or optionally substituted alkyl, aryl, heterocycle, alkyl-aryl or alkyl-heterocycle, or taken together with the nitrogen atom to which they are attached, form an optionally substituted heterocycle; and

n is 0 to 5.

In one embodiment, R₁ is optionally substituted alkyl. In another, it is alkyl (e.g., t-butyl or propyl). In another, it is optionally substituted aryl. In another, it is optionally substituted heterocycle.

In another embodiment, R₂ is hydrogen. In another, it is optionally substituted alkyl (e.g., optionally substituted methyl).

In another embodiment, R₃ is halogen. In another, it is optionally substituted alkyl (e.g., optionally substituted lower alkyl). In another, it is hydroxy.

In another embodiment, R₄ and R₅ are independently hydrogen or optionally substituted alkyl. In another, they are taken together to form optionally substituted pyridine or pyrrolidine.

In another embodiment, n is 0. In another, n is 1. In another, n is 2.

In another embodiment, R₄ and R₅ together with the nitrogen atom to which they are attached do not form 1,4-diaza-bicyclo[3.2.2]nonane. In another embodiment, R₄ and R₅ together with the nitrogen atom to which they are attached do not form piperazine-C(O)-aryl (e.g., piperazine-C(O)-phenyl).

This invention encompasses compounds of formula III-A: 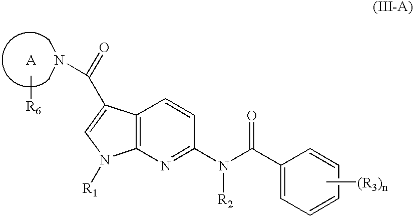
and pharmaceutically acceptable salts and solvates thereof, wherein:

A is a heterocycle;

R₁ is hydrogen or optionally substituted alkyl, aryl, heterocycle, alkyl-aryl or alkyl-heterocycle;

R₂ is hydrogen or optionally substituted alkyl;

each R₃ is independently halogen, amine, hydroxy, alkoxy, or optionally substituted alkyl, aryl or heterocycle;

R₆ is optionally substituted alkyl, aryl, heterocycle, alkyl-aryl or alkyl-heterocycle; and

n is 0 to 5.

In one embodiment, A is pyridine or pyrrolidine.

In one embodiment, R₁ is optionally substituted alkyl. In another, it is alkyl (e.g., t-butyl or propyl). In another, it is optionally substituted aryl. In another, it is optionally substituted heterocycle.

In another embodiment, R₂ is hydrogen. In another, it is optionally substituted alkyl (e.g., optionally substituted methyl).

In another embodiment, R₃ is halogen. In another, it is optionally substituted alkyl (e.g., optionally substituted lower alkyl). In another, it is hydroxy.

In another embodiment, R₆ is optionally substituted alkyl. In another, it is optionally heterocycle. In another, it is a heterocycle (e.g., pyridine or pyrrolidine).

In another embodiment, n is 0. In another, n is 1. In another, n is 2.

In another embodiment, A is not 1,4-diaza-bicyclo[3.2.2]nonane. In another embodiment, A is not piperazine-C(O)-aryl (e.g., piperazine-C(O)-phenyl).

Compounds of the invention may contain one or more stereocenters, and can exist as racemic mixtures of enantiomers or mixtures of diastereomers. This invention encompasses the use of stereomerically pure forms of such compounds, as well as the use of mixtures of those forms. For example, mixtures comprising equal or unequal amounts of the enantiomers of a particular compound of the invention may be used in methods and compositions of the invention. These isomers may be asymmetrically synthesized or resolved using standard techniques such as chiral columns or chiral resolving agents. See, e.g., Jácques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGraw Hill, NY, 1962); and Wilen, S. H., Tables of Resolving Agents and Optical Resolutions, p. 268 (E. L. Eliel, Ed., Univ. of Notre Dame Press, Notre Dame, Ind., 1972).

This invention further encompasses stereoisomeric mixtures of compounds disclosed herein. It also encompasses configurational isomers of compounds disclosed herein, either in admixture or in pure or substantially pure form, such as cis (Z) and trans (E) alkene isomers.

Compounds encompassed by the invention include:
7-tert-butyl-N-isopropyl-4-methyl-2-(4-methylbenzamido)-7H-pyrrolo[2,3-d]pyrimidine-5-carboxamide;
7-tert-butyl-N-isopropyl-2-(4-methylbenzamido)-7H-pyrrolo[2,3-d]pyrimidine-5-carboxamide;
(S)—N-(7-tert-butyl-5-(2-(pyrrolidin-1-ylmethyl)pyrrolidine-1-carbonyl)-7H-pyrrolo[2,3-d]pyrimidin-2-yl)-4-methylbenzamide;
7-tert-butyl-2-(4-methylbenzamido)-N-(pyridin-4-ylmethyl)-7H-pyrrolo[2,3-d]pyrimidine-5-carboxamide;
7-tert-butyl-N,N-dimethyl-2-(4-methylbenzamido)-7H-pyrrolo[2,3-d]pyrimidine-5-carboxamide;
7-tert-butyl-2-(4-methylbenzamido)-N-propyl-7H-pyrrolo[2,3-d]pyrimidine-5-carboxamide;
7-tert-butyl-N-cyclopropyl-2-(4-methylbenzamido)-7H-pyrrolo[2,3-d]pyrimidine-5-carboxamide;
N-(7-tert-butyl-5-(pyrrolidine-1-carbonyl)-7H-pyrrolo[2,3-d]pyrimidin-2-yl)-4-methylbenzamide;
7-tert-butyl-N-ethyl-2-(4-methylbenzamido)-7H-pyrrolo[2,3-d]pyrimidine-5-carboxamide;
7-tert-butyl-2-(4-methylbenzamido)-N-((6-(trifluoromethyl)pyridin-3-yl)methyl)-7H-pyrrolo[2,3-d]pyrimidine-5-carboxamide;
7-tert-butyl-N-methyl-2-(4-methylbenzamido)-7H-pyrrolo[2,3-d]pyrimidine-5-carboxamide;
7-tert-butyl-N-isobutyl-2-(4-methylbenzamido)-7H-pyrrolo[2,3-d]pyrimidine-5-carboxamide;
7-tert-butyl-N-(2-(dimethylamino)ethyl)-2-(4-methylbenzamido)-7H-pyrrolo[2,3-d]pyrimidine-5-carboxamide;
7-tert-butyl-2-(4-methylbenzamido)-N-(pyridin-2-ylmethyl)-7H-pyrrolo[2,3-d]pyrimidine-5-carboxamide;
7-tert-butyl-2-(4-methylbenzamido)-N-(pyridin-3-ylmethyl)-7H-pyrrolo[2,3-d]pyrimidine-5-carboxamide;
N-isopropyl-2-(4-methylbenzamido)-7-propyl-7H-pyrrolo[2,3-d]pyrimidine-5-carboxamide;
2-(4-methylbenzamido)-7-propyl-N-((6-(trifluoromethyl)pyridin-3-yl)methyl)-7H-pyrrolo[2,3-d]pyrimidine-5-carboxamide;
7-tert-butyl-2-(3-fluoro-4-methylbenzamido)-N-isopropyl-7H-pyrrolo[2,3-d]pyrimidine-5-carboxamide;
7-tert-butyl-2-(3-fluoro-4-methylbenzamido)-N-((6-(trifluoromethyl)pyridin-3-yl)methyl)-7H-pyrrolo[2,3-d]pyrimidine-5-carboxamide;
7-tert-butyl-2-(4-ethylbenzamido)-N-((6-(trifluoromethyl)pyridin-3-yl)methyl)-7H-pyrrolo[2,3-d]pyrimidine-5-carboxamide;
7-tert-butyl-2-(4-ethylbenzamido)-N-isopropyl-7H-pyrrolo[2,3-d]pyrimidine-5-carboxamide;
7-tert-butyl-2-(4-ethylbenzamido)-N-(2-methoxyethyl)-7H-pyrrolo[2,3-d]pyrimidine-5-carboxamide;
7-tert-butyl-2-(3-fluoro-4-methylbenzamido)-N-(2-methoxyethyl)-7H-pyrrolo[2,3-d]pyrimidine-5-carboxamide;
7-tert-butyl-2-(3-fluoro-4-methylbenzamido)-N-(pyridin-3-ylmethyl)-7H-pyrrolo[2,3-d]pyrimidine-5-carboxamide;
7-tert-butyl-N-(2-ethoxyethyl)-2-(3-fluoro-4-methylbenzamido)-7H-pyrrolo[2,3-d]pyrimidine-5-carboxamide;
7-tert-butyl-N-ethyl-2-(4-ethylbenzamido)-7H-pyrrolo[2,3-d]pyrimidine-5-carboxamide;
7-tert-butyl-2-(4-ethylbenzamido)-N-isobutyl-7H-pyrrolo[2,3-d]pyrimidine-5-carboxamide;
7-tert-butyl-N-cyclopropyl-2-(4-ethylbenzamido)-7H-pyrrolo[2,3-d]pyrimidine-5-carboxamide;
7-tert-butyl-2-(4-ethylbenzamido)-N-(pyridin-3-ylmethyl)-7H-pyrrolo[2,3-d]pyrimidine-5-carboxamide;
7-tert-butyl-2-(3-fluoro-4-methylbenzamido)-N-isobutyl-7H-pyrrolo[2,3-d]pyrimidine-5-carboxamide;
7-tert-butyl-2-(3-fluoro-4-methylbenzamido)-N-propyl-7H-pyrrolo[2,3-d]pyrimidine-5-carboxamide;
7-tert-butyl-2-(4-ethylbenzamido)-N-propyl-7H-pyrrolo[2,3-d]pyrimidine-5-carboxamide;
7-tert-butyl-N-cyclopropyl-2-(3-fluoro-4-methylbenzamido)-7H-pyrrolo[2,3-d]pyrimidine-5-carboxamide;
7-tert-butyl-N-(2-methoxyethyl)-2-(4-methylbenzamido)-7H-pyrrolo[2,3-d]pyrimidine-5-carboxamide;
7-tert-butyl-N-(2-ethoxyethyl)-2-(4-ethylbenzamido)-7H-pyrrolo[2,3-d]pyrimidine-5-carboxamide;
7-tert-butyl-N-ethyl-2-(3-fluoro-4-methylbenzamido)-7H-pyrrolo[2,3-d]pyrimidine-5-carboxamide;
7-tert-butyl-N-(2-ethoxyethyl)-2-(4-methylbenzamido)-7H-pyrrolo[2,3-d]pyrimidine-5-carboxamide;
7-tert-butyl-N-(1-methoxypropan-2-yl)-2-(4-methylbenzamido)-7H-pyrrolo[2,3-d]pyrimidine-5-carboxamide;
7-tert-butyl-N-isopropyl-2-(4-propylbenzamido)-7H-pyrrolo[2,3-d]pyrimidine-5-carboxamide;
7-tert-butyl-N-(2-ethoxyethyl)-2-(4-propylbenzamido)-7H-pyrrolo[2,3-d]pyrimidine-5-carboxamide;
7-tert-butyl-2-(4-propylbenzamido)-N-(pyridin-3-ylmethyl)-7H-pyrrolo[2,3-d]pyrimidine-5-carboxamide;
7-tert-butyl-N-isopropyl-2-(4-isopropylbenzamido)-7H-pyrrolo[2,3-d]pyrimidine-5-carboxamide;
7-tert-butyl-N-(2-ethoxyethyl)-2-(4-isopropylbenzamido)-7H-pyrrolo[2,3-d]pyrimidine-5-carboxamide;
7-tert-butyl-2-(4-isopropylbenzamido)-N-(pyridin-3-ylmethyl)-7H-pyrrolo[2,3-d]pyrimidine-5-carboxamide;
7-isobutyl-N-isopropyl-2-(4-methylbenzamido)-7H-pyrrolo[2,3-d]pyrimidine-5-carboxamide;
7-isobutyl-2-(4-methylbenzamido)-N-((6-(trifluoromethyl)pyridin-3-yl)methyl)-7H-pyrrolo[2,3-d]pyrimidine-5-carboxamide;
N-isopropyl-2-(4-methylbenzamido)-7-tert-pentyl-7H-pyrrolo[2,3-d]pyrimidine-5-carboxamide;
2-(4-methylbenzamido)-7-tert-pentyl-N-((6-(trifluoromethyl)pyridin-3-yl)methyl)-7H-pyrrolo[2,3-d]pyrimidine-5-carboxamide;
7-tert-butyl-2-(3,4-dimethylbenzamido)-N-isopropyl-7H-pyrrolo[2,3-d]pyrimidine-5-carboxamide;
7-tert-butyl-2-(3,4-dimethylbenzamido)-N-(2-ethoxyethyl)-7H-pyrrolo[2,3-d]pyrimidine-5-carboxamide;
7-tert-butyl-2-(3,4-dimethylbenzamido)-N-(pyridin-3-ylmethyl)-7H-pyrrolo[2,3-d]pyrimidine-5-carboxamide;
4-methyl-N-(7-tert-pentyl-5-(pyrrolidine-1-carbonyl)-7H-pyrrolo[2,3-d]pyrimidin-2-yl)benzamide;
2-(4-methylbenzamido)-7-tert-pentyl-N-(pyridin-3-ylmethyl)-7H-pyrrolo[2,3-d]pyrimidine-5-carboxamide;
N-(2-ethoxyethyl)-2-(4-methylbenzamido)-7-tert-pentyl-7H-pyrrolo[2,3-d]pyrimidine-5-carboxamide;
N-cyclopropyl-2-(4-methylbenzamido)-7-tert-pentyl-7H-pyrrolo[2,3-d]pyrimidine-5-carboxamide;
1-tert-butyl-N-isopropyl-6-(4-methylbenzamido)-1H-pyrazolo[3,4-b]pyridine-3-carboxamide;
1-tert-butyl-6-(4-methylbenzamido)-N-(2-(pyridin-3-yl)ethyl)-1H-pyrazolo[3,4-b]pyridine-3-carboxamide;
1-tert-butyl-6-(4-methylbenzamido)-N-(2-(pyridin-3-yl)ethyl)-1H-pyrazolo[3,4-b]pyridine-3-carboxamide;
(S)—N-(1-tert-butyl-3-(2-isobutylpyrrolidine-1-carbonyl)-1H-pyrazolo[3,4-b]pyridin-6-yl)-4-methylbenzamide;
1-tert-butyl-6-(4-methylbenzamido)-N-(pyridin-3-ylmethyl)-1H-pyrazolo[3,4-b]pyridine-3-carboxamide;
N-(1-tert-butyl-3-(2-(pyridin-2-yl)piperidine-1-carbonyl)-1H-pyrazolo[3,4-b]pyridin-6-yl)-4-methylbenzamide;
1-tert-butyl-N-isobutyl-6-(4-methylbenzamido)-1H-pyrazolo[3,4-b]pyridine-3-carboxamide;
1-tert-butyl-6-(4-methylbenzamido)-N-(pyridin-2-ylmethyl)-1H-pyrazolo[3,4-b]pyridine-3-carboxamide;
1-tert-butyl-N-isobutyl-6-(4-methylbenzamido)-1H-pyrazolo[3,4-b]pyridine-3-carboxamide;
1-tert-butyl-6-(4-methylbenzamido)-N-(pentan-3-yl)-1H-pyrazolo[3,4-b]pyridine-3-carboxamide;
(S)—N-(1-tert-butyl-3-(2-(pyrrolidin-1-ylmethyl)pyrrolidine-1-carbonyl)-1H-pyrazolo[3,4-b]pyridin-6-yl)-4-methylbenzamide;
N-((1H-indol-3-yl)methyl)-1-tert-butyl-6-(4-methylbenzamido)-1H-pyrazolo[3,4-b]pyridine-3-carboxamide;
N-(2-(1H-indol-3-yl)ethyl)-1-tert-butyl-6-(4-methylbenzamido)-1H-pyrazolo[3,4-b]pyridine-3-carboxamide;
1-tert-butyl-6-(4-methylbenzamido)-N-(1-(pyridin-3-yl)ethyl)-1H-pyrazolo[3,4-b]pyridine-3-carboxamide;
1-isopropyl-6-(4-methylbenzamido)-N-(1-(pyridin-3-yl)ethyl)-1H-pyrazolo[3,4-b]pyridine-3-carboxamide;
1-isopropyl-N,N-dimethyl-6-(4-methylbenzamido)-1H-pyrazolo[3,4-b]pyridine-3-carboxamide;
1-tert-butyl-N-isopropyl-6-(6-methylnicotinamido)-1H-pyrazolo[3,4-b]pyridine-3-carboxamide;
6-(4-methylbenzamido)-1-propyl-N-(1-(pyridin-3-yl)ethyl)-1H-pyrazolo[3,4-b]pyridine-3-carboxamide;
1-benzyl-N-isopropyl-6-(4-methylbenzamido)-1H-pyrazolo[3,4-b]pyridine-3-carboxamide;
1-benzyl-N,N-dimethyl-6-(4-methylbenzamido)-1H-pyrazolo[3,4-b]pyridine-3-carboxamide;
1-benzyl-6-(4-methylbenzamido)-N-(2-(pyridin-2-yl)ethyl)-1H-pyrazolo[3,4-b]pyridine-3-carboxamide;
1-benzyl-6-(4-methylbenzamido)-N-(1-(pyridin-3-yl)ethyl)-1H-pyrazolo[3,4-b]pyridine-3-carboxamide;
N,N-dimethyl-6-(4-methylbenzamido)-1-propyl-1H-pyrazolo[3,4-b]pyridine-3-carboxamide;
6-(4-methylbenzamido)-1-propyl-N-(2-(pyridin-2-yl)ethyl)-1H-pyrazolo[3,4-b]pyridine-3-carboxamide;
N-isopropyl-6-(4-methylbenzamido)-1-propyl-1H-pyrazolo[3,4-b]pyridine-3-carboxamide;
1-tert-butyl-N-isopropyl-6-(3-methylbenzamido)-1H-pyrazolo[3,4-b]pyridine-3-carboxamide;
N-isopropyl-6-(4-methylbenzamido)-1-(2,2,2-trifluoroethyl)-1H-pyrazolo[3,4-b]pyridine-3-carboxamide;
6-(4-methylbenzamido)-N-(2-(pyridin-2-yl)ethyl)-1-(2,2,2-trifluoroethyl)-1H-pyrazolo[3,4-b]pyridine-3-carboxamide;
6-(4-methylbenzamido)-N-(1-(pyridin-3-yl)ethyl)-1-(2,2,2-trifluoroethyl)-1H-pyrazolo[3,4-b]pyridine-3-carboxamide;
N-cyclopropyl-6-(4-methylbenzamido)-1-(2,2,2-trifluoroethyl)-1H-pyrazolo[3,4-b]pyridine-3-carboxamide;
N-cyclopropyl-1-isobutyl-6-(4-methylbenzamido)-1H-pyrazolo[3,4-b]pyridine-3-carboxamide;
N-(1-isobutyl-3-(pyrrolidine-1-carbonyl)-1H-pyrazolo[3,4-b]pyridin-6-yl)-4-methylbenzamide;
1-isobutyl-6-(4-methylbenzamido)-N-(1-(pyridin-3-yl)ethyl)-1H-pyrazolo[3,4-b]pyridine-3-carboxamide;
6-(4-methylbenzamido)-1-phenethyl-N-(1-(pyridin-3-yl)ethyl)-1H-pyrazolo[3,4-b]pyridine-3-carboxamide;
N-cyclopropyl-6-(4-methylbenzamido)-1-phenethyl-1H-pyrazolo[3,4-b]pyridine-3-carboxamide;
4-methyl-N-(1-phenethyl-3-(pyrrolidine-1-carbonyl)-1H-pyrazolo[3,4-b]pyridin-6-yl)benzamide;
N-(1-(cyclobutylmethyl)-3-(pyrrolidine-1-carbonyl)-1H-pyrazolo[3,4-b]pyridin-6-yl)-4-methylbenzamide;
1-(cyclobutylmethyl)-N-cyclopropyl-6-(4-methylbenzamido)-1H-pyrazolo[3,4-b]pyridine-3-carboxamide;
N-cyclopropyl-1-isopentyl-6-(4-methylbenzamido)-1H-pyrazolo[3,4-b]pyridine-3-carboxamide;
N-(1-isopentyl-3-(pyrrolidine-1-carbonyl)-1H-pyrazolo[3,4-b]pyridin-6-yl)-4-methylbenzamide;
1-(cyclobutylmethyl)-6-(4-methylbenzamido)-N-pentyl-1H-pyrazolo[3,4-b]pyridine-3-carboxamide;
1-tert-butyl-N-cyclopropyl-6-(4-methylbenzamido)-1H-pyrazolo[3,4-b]pyridine-3-carboxamide;
1-tert-butyl-N-isopropyl-6-(4-methylbenzamido)-1H-pyrrolo[2,3-b]pyridine-3-carboxamide;
N-(1-tert-butyl-3-(pyrrolidine-1-carbonyl)-1H-pyrrolo[2,3-b]pyridin-6-yl)-4-methylbenzamide;
1-tert-butyl-6-(4-methylbenzamido)-N-(pentan-3-yl)-1H-pyrrolo[2,3-b]pyridine-3-carboxamide;
1-tert-butyl-N,N-diethyl-6-(4-methylbenzamido)-1H-pyrrolo[2,3-b]pyridine-3-carboxamide;
1-tert-butyl-N-isobutyl-6-(4-methylbenzamido)-1H-pyrrolo[2,3-b]pyridine-3-carboxamide;
1-tert-butyl-N-cyclopropyl-6-(4-methylbenzamido)-1H-pyrrolo[2,3-b]pyridine-3-carboxamide;
1-tert-butyl-6-(4-methylbenzamido)-N-(pyridin-2-yl)-1H-pyrrolo[2,3-b]pyridine-3-carboxamide;
1-tert-butyl-6-(4-methylbenzamido)-N-(pyridin-4-yl)-1H-pyrrolo[2,3-b]pyridine-3-carboxamide;
1-tert-butyl-N-ethyl-6-(4-methylbenzamido)-1H-pyrrolo[2,3-b]pyridine-3-carboxamide;
1-tert-butyl-N-(2-ethoxyethyl)-6-(4-methylbenzamido)-1H-pyrrolo[2,3-b]pyridine-3-carboxamide;
N-benzyl-1-tert-butyl-6-(4-methylbenzamido)-1H-pyrrolo[2,3-b]pyridine-3-carboxamide;
1-tert-butyl-6-(4-methylbenzamido)-N-((3-methylpyridin-2-yl)methyl)-1H-pyrrolo[2,3-b]pyridine-3-carboxamide;
1-tert-butyl-6-(4-methylbenzamido)-N-(1-(pyridin-3-yl)ethyl)-1H-pyrrolo[2,3-b]pyridine-3-carboxamide;
1-tert-butyl-N,N-dimethyl-6-(4-methylbenzamido)-1H-pyrrolo[2,3-b]pyridine-3-carboxamide;
(S)—N-sec-butyl-1-tert-butyl-6-(4-methylbenzamido)-1H-pyrrolo[2,3-b]pyridine-3-carboxamide;
1-tert-butyl-6-(4-methylbenzamido)-N-(1-(pyridin-4-yl)ethyl)-1H-pyrrolo[2,3-b]pyridine-3-carboxamide;
(S)-1-tert-butyl-N-(1-methoxypropan-2-yl)-6-(4-methylbenzamido)-1H-pyrrolo[2,3-b]pyridine-3-carboxamide;
(R)—N-(1-tert-butyl-3-(2-(methoxymethyl)pyrrolidine-1-carbonyl)-1H-pyrrolo[2,3-b]pyridin-6-yl)-4-methylbenzamide;
1-tert-butyl-6-(4-methylbenzamido)-N-propyl-1H-pyrrolo[2,3-b]pyridine-3-carboxamide;
1-tert-butyl-N-cyclobutyl-6-(4-methylbenzamido)-1H-pyrrolo[2,3-b]pyridine-3-carboxamide;
(R)-1-tert-butyl-6-(4-methylbenzamido)-N-(3-methylbutan-2-yl)-1H-pyrrolo[2,3-b]pyridine-3-carboxamide;
1-tert-butyl-6-(4-methylbenzamido)-N-(thiazol-2-ylmethyl)-1H-pyrrolo[2,3-b]pyridine-3-carboxamide;
(R)—N-(1-tert-butyl-3-(2-((dimethylamino)methyl)pyrrolidine-1-carbonyl)-1H-pyrrolo[2,3-b]pyridin-6-yl)-4-methylbenzamide;
1-tert-butyl-N-(cyclopropylmethyl)-6-(4-methylbenzamido)-1H-pyrrolo[2,3-b]pyridine-3-carboxamide;
N-(1-tert-butyl-3-(morpholine-4-carbonyl)-1H-pyrrolo[2,3-b]pyridin-6-yl)-4-methylbenzamide;
1-tert-butyl-6-(4-methylbenzamido)-N-(tetrahydro-2H-pyran-4-yl)-1H-pyrrolo[2,3-b]pyridine-3-carboxamide;
(S)-1-tert-butyl-N-(2-hydroxy-1-phenylethyl)-6-(4-methylbenzamido)-1H-pyrrolo[2,3-b]pyridine-3-carboxamide;
N-(2-tert-butoxyethyl)-1-tert-butyl-6-(4-methylbenzamido)-1H-pyrrolo[2,3-b]pyridine-3-carboxamide;
1-tert-butyl-6-(4-methylbenzamido)-N-(1-methylpiperidin-4-yl)-1H-pyrrolo[2,3-b]pyridine-3-carboxamide;
1-tert-butyl-N-(furan-2-yl)-6-(4-methylbenzamido)-1H-pyrrolo[2,3-b]pyridine-3-carboxamide;
(R)-1-tert-butyl-N-(hexan-2-yl)-6-(4-methylbenzamido)-1H-pyrrolo[2,3-b]pyridine-3-carboxamide;
1-tert-butyl-6-(4-methylbenzamido)-N-(oxazol-2-ylmethyl)-1H-pyrrolo[2,3-b]pyridine-3-carboxamide;
(S)—N-(1-methoxypropan-2-yl)-6-(4-methylbenzamido)-1-neopentyl-1H-pyrrolo[2,3-b]pyridine-3-carboxamide;
(R)-1-tert-butyl-6-(4-ethylbenzamido)-N-(1-methoxypropan-2-yl)-1H-pyrrolo[2,3-b]pyridine-3-carboxamide;
(R)-6-(4-ethylbenzamido)-1-isobutyl-N-(1-methoxypropan-2-yl)-1H-pyrrolo[2,3-b]pyridine-3-carboxamide;
1-tert-butyl-N-(1-methoxypropan-2-yl)-6-(4-methylbenzamido)-1H-pyrrolo[2,3-b]pyridine-3-carboxamide; and
(R)-1-tert-butyl-6-(N,4-dimethylbenzamido)-N-(1-methoxypropan-2-yl)-1H-pyrrolo[2,3-b]pyridine-3-carboxamide.

Preferred compounds of the invention are specific proline transporter inhibitors. Particular specific proline transporter inhibitors have a PTIC₅₀ of less than about 150, 125, 100, 75, 50 or 25 nM.

Some compounds inhibit the murine Na⁺-dependent proline transporter, as determined by the method described in the Examples below, with an IC₅₀ of less than about 150, 125, 100, 75, 50 or 25 nM.

Some compounds do not significantly inhibit the dopamine transporter. For example, some specific proline transporter inhibitors inhibit the dopamine transporter with an IC₅₀ of greater than about 0.5, 1, 2.5, 5, or 10 μM as determined using the assay described in the Examples below.

Some compounds do not significantly inhibit the glycine transporter. For example, some specific proline transporter inhibitors inhibit the glycine transporter with an IC₅₀ of greater than about 0.5, 1, 2.5, 5, or 10 μM as determined using the assay described in the Examples below.

4.3. Synthesis

Compounds of the invention (i.e., compounds disclosed herein) may be obtained or prepared using methods known in the art, as well as those described herein.

For example, pyrrolopyrimidine compounds can generally be prepared as shown below in Scheme 1:

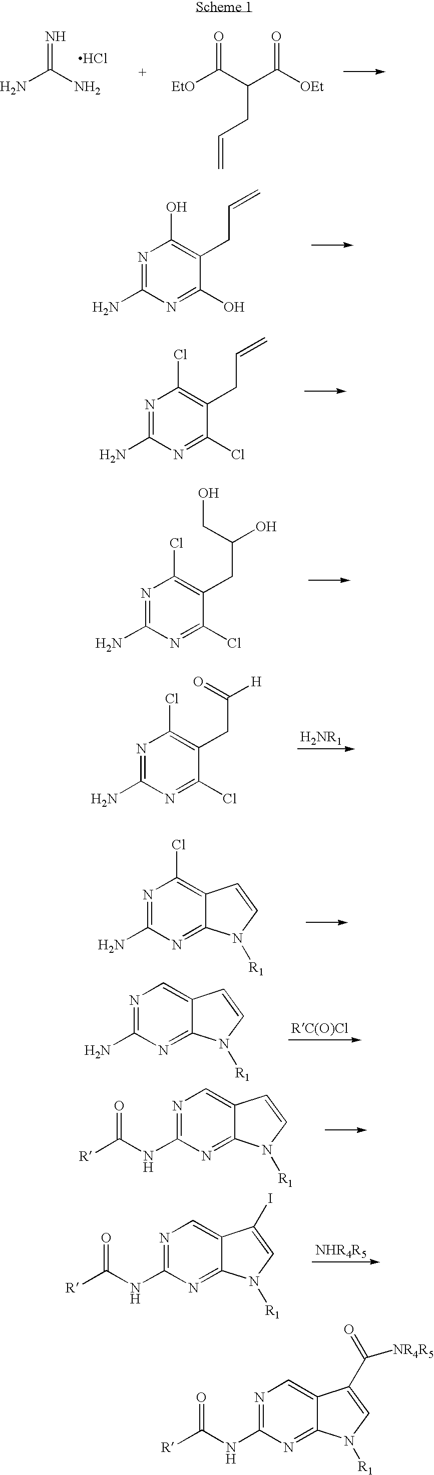

In this approach, 5-allyl-2-amino-pyrimidine-4,6-diol is prepared by the reaction of guanidine with 2-allyl-malonic acid diethyl ester (e.g., in base). The diol is converted to the corresponding di-chloride (e.g., with POCl₃), which is then oxidized (e.g., with OsO₄) to afford 3-(2-amino-4,6-dichloro-pyrimidin-5-yl)-propane-1,2-diol, which is subsequently converted to (2-amino-4,6-dichloro-pyrimidin-5-yl)-acetaldehyde (e.g., with Pb(OAc)₄). The aldehylde is cyclized to obtain a substituted 4-chloro-pyrrolopyrimidine. The chlorine is removed (e.g., with H₂, Pd/C), and the resulting compound is reacted with the desired acid chloride, then iodinated, and finally reacted with the desired amine to obtain the final product.

Pyrrolopyridine compounds can generally be prepared as shown below in Scheme 2:

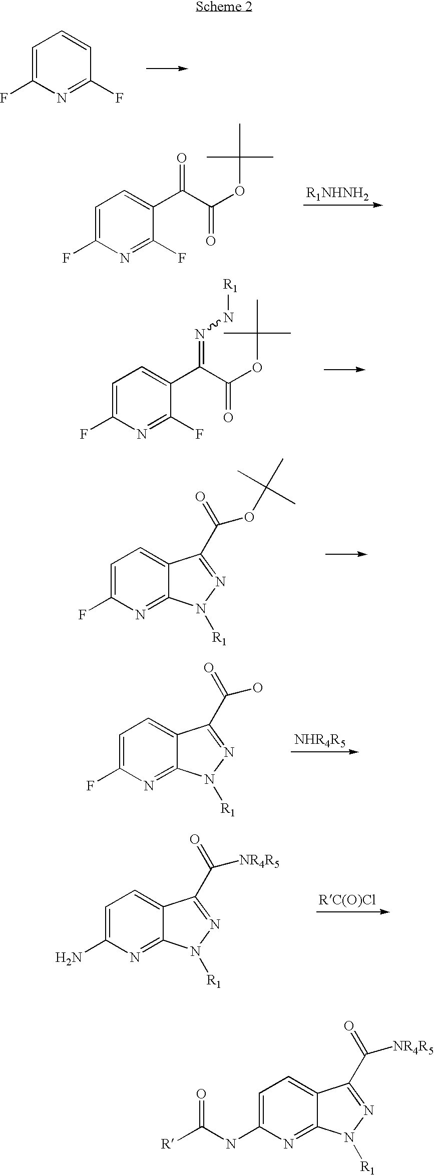

In this approach, 2,6-difluoro-pyridine is reacted with oxalic acid di-tert-butyl ester to afford (2,6-difluoro-pyridin-3-yl)-oxo-acetic acid tert-butyl ester. This is converted to the desired (2,6-difluoro-pyridin-3-yl)-hydrazono-acetic acid tert-butyl ester, which is subsequently cyclized to afford the corresponding 6-fluoro-1H-pyrazolo[3,4-b]pyridine-3-carboxylic acid tert-butyl ester. The tert-butyl ester is removed to yield the corresponding acid, which is reacted with the appropriate amine to afford the desired amide. The amide is reacted with the desired acid chloride to obtain the final product.

Pyrazolopyrimidine compounds can generally be prepared as shown in Scheme 3:

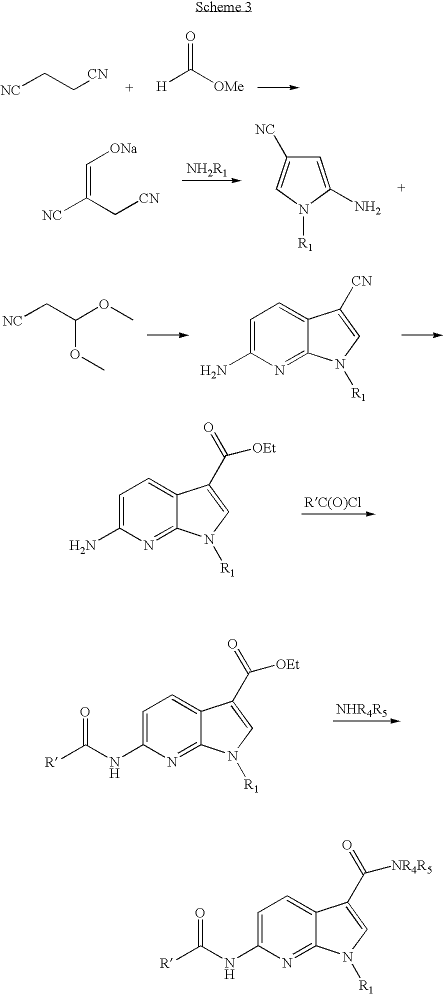

In this approach, succinonitrile is reacted with formic acid methyl ester to afford 2,3-dicyano-propen-1-ol sodium, with is reacted with an amine to yield the desired N-substituted 5-amino-1H-pyrrole-3-carbonitrile. The pyrrole is reacted with 3,3-dimethoxy-propionitrile in acidic conditions to afford a 6-amino-1H-pyrrolo[2,3-b]pyridine-3-carbonitrile, which is converted into the corresponding ethyl ester (e.g., with H₂SO₄ in EtOH). The ethyl ester is next reacted with the desired acid chloride, and finally reacted with the desired amine to yield the final product.

4.4. Methods of Treatment

One embodiment of this invention encompasses a method of inhibiting a proline transporter, which comprises contacting a proline transporter (in vitro or in vivo) with a sufficient amount of a compound of the invention. Preferred proline transporters are encoded by the human gene SLC6A7, the murine ortholog thereof, or a nucleic acid molecule that encodes a proline transporter and that hybridizes under standard conditions to the full length of either.

Another embodiment encompasses a method of improving the cognitive performance of a human patient, which comprises administering to the patient an effective amount of a compound of the invention. Examples of improved cognitive performance include enhanced learning (e.g., learning more quickly), improved comprehension, improved reasoning, and improved short- and/or long-term memory.

Another embodiment encompasses a method of treating, managing or preventing a disease or disorder in a human patient, which comprises administering to the patient a therapeutically or prophylactically effective amount of a compound of the invention. Examples of diseases and disorders include Alzheimer's disease, autism, cognitive disorders (e.g., difficulty in thinking, reasoning, or problem solving), dementia, learning disorders (e.g., dyslexia, dyscalculia, dysgraphia, dysphasia, dysnomia), and short- and long-term memory loss. Additional disorders include adverse sequelae of brain damage caused by, for example, oxygen starvation, traumatic injury or stroke.

4.5. Pharmaceutical Compositions

This invention encompasses pharmaceutical compositions and dosage forms comprising compounds of the invention as their active ingredients. Pharmaceutical compositions and dosage forms of this invention may optionally contain one or more pharmaceutically acceptable carriers or excipients. Certain pharmaceutical compositions are single unit dosage forms suitable for oral, topical, mucosal (e.g., nasal, pulmonary, sublingual, vaginal, buccal, or rectal), parenteral (e.g., subcutaneous, intravenous, bolus injection, intramuscular, or intra-arterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, oral administration may require enteric coatings to protect the active ingredient from degradation within the gastrointestinal tract. In another example, the active ingredient may be administered in a liposomal formulation to shield it from degradative enzymes, facilitate transport in circulatory system, and/or effect delivery across cell membranes to intracellular sites.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

### 5. EXAMPLES

Some examples of various aspects of the invention are described below. In the synthetic examples, the following HPLC conditions were used unless otherwise noted: Solvent A—90% water/10% methanol/0.1% TFA; Solvent B—20% water/90% methanol/0.1% TFA; Column—YMC-Pack ODS-A 4.6×33 mm; gradient time: 4 min; flow rate: 3 ml/minute.

5.1. SLC6A7-Deficient Mice

To determine the effect of inhibiting the Na⁺-dependent proline transporter, mice homozygous for a genetically engineered mutation in the murine ortholog of the human SLC6A7 gene (“knockout” or “KO” mice) were generated using correspondingly mutated ES cell clones from the OMNIBANK collection of mutated murine ES cell clones (see generally U.S. Pat. No. 6,080,576).

Mice that were heterozygous, homozygous, or wildtype for the mutated allele were produced by breeding heterozygous animals capable of germline transmission of the mutant allele. The mutated allele assorted according to standard Mendelian genetics. The mice were subjected to a battery of medical and behavioral tests, including those described below.

5.1.1. Trace Conditioning

Trace aversive conditioning measures a form of classical conditioning with temporal separation between the end of a conditioned stimulus (CS) (in this case an 80 db tone) and the onset of an unconditioned stimulus (US) (in this case a 0.7 mA electric current) that are separated by a temporal “trace” (approximately 30 seconds). This assay measures higher-order learning (usually associated with hippocampal finction or the cortex) by determining how rapidly the test subjects learn to associate the US with CS. The test animals are scored by calculating the percent freezing time as determined by comparing the difference between percent freezing post-CS and the percent freezing pre-CS.

Both male and female animals that were homozygous for the mutation in the murine ortholog of the SLC6A7 gene displayed significantly higher freezing percentages (approximately 50 percent for an average of 16 test animals) as compared to their wildtype control counterparts (approximately 30 percent for an average of 16 control animals). These results indicate that homozygous mutant animals perform significantly better in this well established test for cognitive performance.

5.1.2. Water Maze

The Morris water maze used a circular pool 2 meters in diameter and 40 cm in depth. See, e.g., Morris, 1984, J. Neurosci. Methods 11:47-60, Guillou et al., 1999, J. Neurocsci. 19:6183-90. The pool was filled to a depth of 30 cm with water at a temperature of 24-26° C., made opaque by the addition of non-toxic water-based paint. The “escape” platform was about 30 cm high with a plastic disc 18 cm in diameter on top. The platform was placed about 0.5 cm below the water surface. The mouse was released into the pool facing the wall from one of 4 start positions labeled as N (North), S (South), W (West) or E (East). A videotracking system comprising the camera and the WaterMaze image software (Actimetrics, Inc.) divided the pool into four equal quadrants designated as SE, SW, NE, and NW. The software calculates the latency to reach platform, distance to the platform, time spent in each quadrant, swimming speed, and other parameters.

Each trial lasted until the mouse climbed onto the platform or 90 seconds had elapsed. If the mouse did not reach the platform in 90 seconds, the experimenter took it out of the water and gently placed it on the platform. At the end of each trial the mouse remained on the platform for further 20 seconds. There were 4 trials with platform per day with 8-12 min inter-trial intervals. During the inter-trial interval the mouse was kept in a clean cage under a heat lamp.

Typically one of two basic protocols were used: the first includes visible and hidden platform phases, and the second only uses a hidden platform phase; both protocols end with a two day reversal phase.

The visible phase generally precedes the hidden platform phase. In the visible phase, the pool was surrounded with white curtains in order to hide all external-maze cues/references. During this phase, the platform was made visible with a metal cylinder 8 cm h×3 cm, which was put on the platform. The start position was the same on each trial, while platform location was randomly changed during the trials. This phase lasted for approximately 3 days.

In the hidden platform phase, the platform was no longer marked and the curtains were removed. A variety of extra-maze cues were optionally placed around the pool. Here the start position was changed every trial, while the platform remained in the same location. This phase typically lasted about seven days.

Probe trials were run before the training trials on days one and five of the hidden phase, and on day one of the visible phase, and also after the last trial on day three of the visible phase. During the probe trial, the platform was removed from the pool and the mouse was placed in the pool facing the wall in the quadrant opposite from the platform quadrant. The mouse swam for 60 sec and the percentage of time spent in each quadrant was recorded.

In the reversal phase, on each of two days, five trials were run. During the first trial the platform location was the same as it was in the hidden phase. In the next four trials, the platform was moved to the opposite quadrant. On the following day the platform was there on first trial and then again moved to the left or right adjoining quadrant for the last 4 trials. The start position was always kept opposite to the platform location.

When the above methods were used with SLC6A7 KO mice (n=12) and WT (n=7) controls, mice were first subjected to the visible platform task. Repeated measures (RM) and analysis of variance (ANOVA) were used to analyze genotype effect on the latency to reach platform over 11 trials.

The trial effect was F(10, 170)=8.57, p<0.001; the Genotype effect: F(1, 17)=0.65, p<0.43, interaction Genotype x Trial: F(10, 170)=0.42, p<0.93. Initially, there was no difference between WT and KO subjects, but a significant decrease in the latency over trails was observed.

When the trials progressed to the hidden platform task, RM ANOVA revealed a significant effect of the trials on the latency to reach platform: F(19, 323)=7.2, p<0.001. There was also a significant effect of genotype on same parameter: F(1, 17)=8.0, p<0.012; interaction Genotype x Trials was F(19, 323)=1.16, p<0.29. Overall, KO subjects had significantly shorter latencies to platform. No significant difference in swimming speed was detected so faster swimming did not account for the faster performance by the KO animals.

During the reversal phase, RM ANOVA was run on 4 trials with the platform switched to another quadrant on each of two days. On both days of reversal phase effect of trials was significant: Fs(3, 51)>6.4, p<0.001 indicating that both genotypes relearn well. However, there was no significant difference between them on each day of reversal: Fs(1, 17)<0.75, ps>0.39, although KO mice did tend to reach the platform faster.

During probe trials, the percent of time spent in each quadrant was compared with 25% chance for WT and KO mice by non-parametric Mann-Whitney test. The first two probe trials run before hidden phase the percent time was not different from chance in each quadrant for both genotypes. In the third probe trial run on the fifth day of hidden phase, the platform quadrant time was significantly different from chance for WT[p<0.05] and KO mice[p<0.001]; and the opposite quadrant time was significantly different for KO mice[p<0.001].

The above data indicate that KO mice learned the hidden platform task more quickly than WT animals. The data further establish that the observed difference cannot be explained by differences in visual abilities or swimming speed between genotypes.

### 5.2. Preparation of (2-Amino-4,6-dichloro-pyrimidin-5-yl)-acetaldehyde

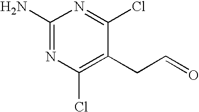

5-Allyl-2-amino-pyrimidine-4,6-diol (3): Under a nitrogen atmosphere, NaOEt was prepared by dissolving sodium metal (4.30 g, 187 mmol) into 100 ml of EtOH. At 0° C. guanidine (1) (4.80 g, 50.2 mmol) was added and the solution was stirred for 10 minutes. Diethyl allyl malonate (2) (10 ml, 50.4 mmol) was added dropwise after which the mixture was allowed to warm to room temperature. After stirring for 65 hours the reaction was quenched with 20 ml of concentrated HCl. The precipitate was filtered and washed with water and ethanol yielding pyrimidine 3 (4.29 g, 51%) as a white solid: ¹H NMR (300 MHz, (CD₃)₂SO)δ 10.32 (s, 2H) 6.37 (s, 2H), 5.81-5.68 (m, 1H), 4.91-4.78 (m, 2H), and 2.85 (d, J=6.0 Hz, 2H); m/z calcd. for C7H9N3O2:167.17. found: (M+H)⁺ 168.10. HPLC retention time=0.677 min (gradient of solvent B—0 to 100%; wavelength 220 nM).

5-Allyl-4,6-dichloro-pyrimidin-2-ylamine (4): Under a nitrogen atmosphere, pyrimidine 3 (1.027 g, 6.15 mmol) was added to 10 ml of POCl₃. The mixture was refluxed at 110° C. After stirring for 30 min the POCl₃ was removed with the rotary evaporator. The crude mixture was very slowly quenched with 15 ml of hot distilled water. The aqueous mixture was extracted twice with CH₂Cl₂. The organic layers were combined, washed with a 1:1 mixture of saturated NaHCO_{3(aq)}/brine, dried over MgSO₄ and concentrated to yield pyrimidine 4 (320 mg, 26%) as a beige solid: ¹H NMR (300 MHz, (CDCl₃) δ 5.93-5.80 (m, 1H), 5.20-5.06 (m, 2H), and 3.52-3.49 (m, 2H); m/z calcd. for C7H7Cl2N3: 204.06. found: 204.00. HPLC retention time=3.631 min (gradient of solvent B—0 to 100%; wavelength 220 nM).

3-(2-Amino-4,6-dichloro-pyrimidin-5-yl)-propane-1,2-diol (5): To a stirring solution of pyrimidine 4 (320 mg, 1.58 mmol) in 15 ml of THF and 3 ml of water was added NMO (370 mg, 3.15 mmol) and then a few crystals of osmium tetroxide. The reaction flask was covered to block exposure to light and the mixture was stirred at room temperature. After 12 h of stirring 10 ml of an aqueous solution of NaHSO₃ (500 mg) was added to the mixture and allowed to stir for a few minutes. The mixture was filtered and the precipitate was washed with water and then triturated with Et₂O to yield some diol 5 as a white solid. The filtrate was extracted three times with EtOAc. The organic phases were combined, washed with brine, dried over MgSO₄ and concentrated in vacuo to yield more diol 5 as a white solid that was combined with the precipitated solid (329 mg, 88%): ¹H NMR (300 MHz, (CD₃)₂SO) δ 7.29 (s, 2H), 4.70 (d, J=5.1 Hz, 1H), 4.62 (t, J=5.9 Hz, 1H), 3.75-3.65 (m, 1H), 2.77-2.60 (m, 2H); m/z calcd. for C7H9Cl2N3O2: 238.07. found: 238.10. HPLC retention time=1.703 min (gradient of solvent B—0 to 100%; wavelength 220 nM).

(2-Amino-4,6-dichloro-pyrimidin-5-yl)-acetaldehyde (6): Under a nitrogen atmosphere, to a stirring suspension of diol 5 (329 mg, 1.39 mmol) in 10 ml of THF and 5 ml of methanol at 0° C. was added lead acetate (700 mg, 1.58 mmol). The mixture was stirred at 0° C. for 1 h and then diluted with EtOAc. The mixture was filtered through Celite. The filtrate was washed three times with a mixture of 1:1 saturated NaHCO_{3(aq)}/brine, dried over MgSO₄ and then concentrated to give aldehyde 6 (253 mg, 88%) as a white solid: m/z calcd. for C7H9Cl2N3O2: 206.03. found: 206.00. HPLC retention time=2.048 min (gradient of solvent B—0 to 100%; wavelength 220 nM).

### 5.3. Preparation of N-(7-tert-Butyl-5-iodo-7H-pyrrolo[2,3-d]pyrimidin-2-yl)-4-methyl-benzamide

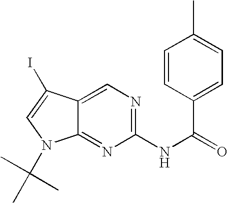

7-tert-Butyl-4-chloro-7H-pyrrolo[2,3-d]pyrimidin-2-ylamine (7): In a sealed pressure vessel aldehyde 6 (253 mg, 1.23 mmol) was suspended in 15 ml of n-butanol. To this mixture was added tert-butyl amine (0.30 ml, 2.78 mmol). After stirring for 5 min at room temperature, triethylamine (0.80 ml, 5.56 mmol) was added and the mixture was stirred in the sealed tube at 115° C. After 14 h the n-butanol was removed with the rotary evaporator. The crude product was purified by silica gel column chromatography (100% DCM) to give chloropyrrolopyrimidine 7 (170 mg, 62%): ¹H NMR (300 MHz, (CDCl₃) δ 7.05 (d, J=3.6 Hz, 1H), 6.35 (d, J=3.9 Hz, 1H), 4.90 (bs, 2H); m/z calcd. for C10H13ClN4: 224.69. found: 225.10. HPLC retention time=3.848 min (gradient of solvent B—0 to 100%; wavelength 220 nM).

7-tert-Butyl-7H-pyrrolo[2,3-d]pyrimidin-2-ylamine (8): Chloropyrrolopyrimidine 7 (308 mg, 1.38 mmol) was dissolved in 25 ml of methanol. To this was added 3 ml concentrated ammonia and a catalytic amount of palladium on carbon. The mixture was stirred under a hydrogen atmosphere at room temperature. After stirring for 2.5 h the mixture was filtered through Celite and the filtrate was concentrated. The crude product was passed through a plug of silica gel to yield pyrrolopyrimidine 8 (240 mg, 92%) as a yellow solid: m/z calcd. for C10H14N4: 190.25. found: 191.00. HPLC retention time=2.477 min (gradient of solvent B—0 to 100%; wavelength 220 nM).

N-(7-tert-Butyl-7H-pyrrolo[2,3-d]pyrimidin-2-yl)-4-methyl-benzamide (10): Under a nitrogen atmosphere, pyrrolopyrimidine 8 (199 mg, 1.05 mmol) was dissolved in 15 ml of THF. To this solution was added triethylamine (0.60 ml, 4.21 mmol) and 4-methylbenzoyl chloride (9) (0.42 ml, 3.16 mmol). The mixture was stirred at room temperature. After 45 min the mixture was diluted with a saturated solution of NaHCO_{3(aq)} and methylene chloride. The layers were separated and the aqueous portion was extracted twice more with methylene chloride. The organic phases were combined, dried over MgSO₄ and then concentrated. To a stirring solution of the residue in 15 ml of methanol was added 3 ml of a 2 N solution of NaOH_{(aq)}. After stirring for 1.5 h the mixture was diluted with a saturated solution of NaHCO_{3(aq)} and EtOAc. The layers were separated and the aqueous portion was extracted once more with EtOAc. The organic layers were combined, dried over MgSO₄ and then concentrated. The crude product was purified by silica gel colunm chromatography (EtOAc:hexanes, 1:4) to give the product 10 as a beige solid (222 mg, 69%): m/z calcd. for C18H20N4O: 308.39. found: 309.05. HPLC retention time=3.686 min (gradient of solvent B—0 to 100%; wavelength 220 nM).

N-(7-tert-Butyl-5-iodo-7H-pyrrolo[2,3-d]pyrimidin-2-yl)-4-methyl-benzamide (11): To a solution of the amide 10 (222 mg, 0.72 mmol) in THF was added NIS (202 mg, 1.25 mmol). The reaction flask was covered to block exposure to light and the mixture was stirred at room temperature. After 17.5 h the solvent was removed in vacuo and the residue was diluted with a saturated solution of NaHCO_{3(aq)} and methylene chloride. The layers were separated and the aqueous portion was extracted three times more with methylene chloride. The organic phases were combined, dried over MgSO₄ and then concentrated. The crude product was purified by silica gel column chromatography (EtOAc:hexanes, 1:5) to yield the iodinated product 11 (185 mg, 59%) as a brown solid: m/z calcd. for C18H19IN4O: 434.28. found: 435.00. HPLC retention time=4.031 min (gradient of solvent B—0 to 100%; wavelength 220 nM).

### 5.4. Preparation of 7-tert-Butyl-2-(4-methyl-benzoylamino)-7H-pyrrolo[2,3-d]pyrimidine-5-carboxylic acid ethylamide

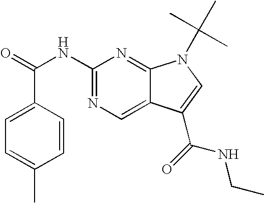

Under a blanket of nitrogen and in a scintillating vial, amide 11 (35 mg, 0.081 mmol) was dissolved in 1 ml of DMF. The solution was degassed using nitrogen and then trans-dichlorobis(triphenylphosphine)palladium (5.6 mg, 0.0081 mmol) was added. After degassing with nitrogen once more the mixture was bubbled through with carbon monoxide for 3 min. A 2 M solution of ethylamine in THF (0.081 ml, 0.162 mmol) was added to the mixture and the vial was sealed. The mixture was stirred at 80° C. After stirring for 12 h the mixture was diluted with EtOAc and filtered through Celite. The filtrate was concentrated and the residue was purified by prep-HPLC to yield the title compound (19 mg, 61%) as a white solid: ¹H NMR (300 MHz, MeOD) δ 9.34 (s, 1H), 8.49 (s, 1H), 7.99 (d, J=9.0 Hz, 2H), 7.41 (d, J=8.0 Hz, 2H), 3.44 (q, J=7.5 Hz, 2H), 2.46 (s, 3H), 1.87 (s, 9H), and 1.26 (t, J=7.2 Hz, 3H); m/z calcd. for C21H25N5O2: 379.47. found: 380.25. HPLC retention time=3.620 min (gradient of solvent B—0 to 100%; wavelength 220 nM).

### 5.5. Preparation of 7-tert-Butyl-2-(4-methyl-benzoylamino)-7H-pyrrolo[2,3-d]pyrimidine-5-carboxylic acid (pyridin-3-ylmethyl)-amide

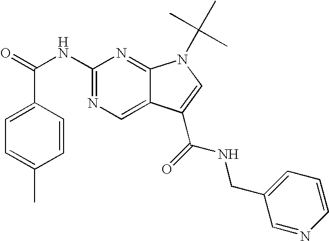

Under a blanket of nitrogen and in a scintillating vial, amide 11 (35 mg, 0.081 mmol) was dissolved in 1 ml of DMF. The solution was degassed using nitrogen and then trans-dichlorobis(triphenylphosphine)palladium (5.6 mg, 0.0081 mmol) was added. After degassing with nitrogen once more the mixture was bubbled through with carbon monoxide for 3 min. To the solution was added 3-(aminomethyl)pyridine (0.017 ml, 0.162 mmol) and the vial was sealed. The mixture was stirred at 80° C. After stirring for 12 h the mixture was diluted with EtOAc and filtered through Celite. The filtrate was concentrated and the residue was purified by prep-HPLC to yield the title compound (25 mg, 70%) as a white solid: ¹H NMR (300 MHz, MeOD) δ 9.34 (s, 1H), 8.79 (s, 1H) 8.67 (d, J=5.1 Hz, 1H), 8.49 (s, 1H), 8.37 (d, J=8.0 Hz, 1H), 7.97 (d, J=8.6 Hz, 2H), 7.84 (dd, J=5.9, 2.4 Hz, 1H), 7.40 (d, J=8.3 Hz, 2H), 4.73 (s, 2H), 2.46 (s, 3H), and 1.87 (s, 9H); m/z calcd. for C25H26N6O2: 442.52. found: 443.40. HPLC retention time=3.196 min (gradient of solvent B—0 to 100%; wavelength 220 nM).

### 5.6. Preparation of 7-tert-Butyl-2-(4-methyl-benzoylamino)-7H-pyrrolo[2,3-d]pyrimidine-5-carboxylic acid (pyridin-2-ylmethyl)-amide

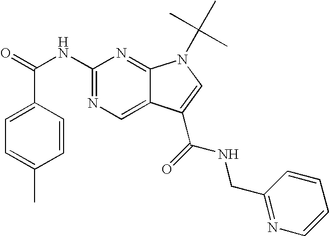

Under a blanket of nitrogen and in a scintillating vial, amide 11 (35 mg, 0.081 mmol) was dissolved in 1 ml of DMF. The solution was degassed using nitrogen and then trans-dichlorobis(triphenylphosphine)palladium (5.6 mg, 0.0081 mmol) was added. After degassing with nitrogen once more the mixture was bubbled through with carbon monoxide for 3 min. To the solution was added 2-(aminomethyl)pyridine (0.017 ml, 0.162 mmol) and the vial was sealed. The mixture was stirred at 80° C. After stirring for 12 h the mixture was diluted with EtOAc and filtered through Celite. The filtrate was concentrated and the residue was purified by prep-HPLC to yield the title compound (19 mg, 52%) as a beige solid: ¹H NMR (300 MHz, MeOD) δ 9.34 (s, 1H), 8.65 (d, J=4.5 Hz, 1H), 8.57 (s, 1H), 8.19 (td, J=7.8, 1.5 Hz, 1H), 7.98 (d, J=8.1 Hz, 2H), 7.78 (d, J=7.8 Hz, 1H), 7.64 (app t, J=6.3 Hz, 1H), 7.41 (d, J=7.8 Hz, 2H), 4.81 (s, 2H), 2.46 (s, 3H), and 1.89 (s, 9H); m/z calcd. for C25H26N6O2: 442.52. found: 443.35. HPLC retention time=3.211 min (gradient of solvent B—0 to 100%; wavelength 220 nM).

### 5.7. Preparation of 7-tert-Butyl-2-(4-methyl-benzoylamino)-7H-pyrrolo[2,3-d]pyrimidine-5-carboxylic acid (2-dimethylamino-ethyl)-amide

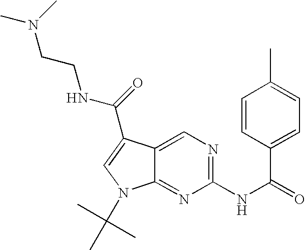

Under a blanket of nitrogen and in a scintillating vial, amide 11 (35 mg, 0.081 mmol) was dissolved in 1 ml of DMF. The solution was degassed using nitrogen and then trans-dichlorobis(triphenylphosphine)palladium (5.6 mg, 0.0081 mmol) was added. After degassing with nitrogen once more the mixture was bubbled through with carbon monoxide for 3 min. N,N-dimethyl ethylene diamine (0.014 ml, 0.162 mmol) was added to the mixture and the vial was sealed. The mixture was stirred at 80° C. After stirring for 12 h the mixture was diluted with EtOAc and filtered through Celite. The filtrate was concentrated and the residue was purified by prep-HPLC to yield the title compound (8.9 mg, 26%) as a beige solid: ¹H NMR (300 MHz, MeOD) δ 9.34 (s, 1H), 8.36 (s, 1H), 7.95 (d, J=8.3 Hz, 2H), 7.39 (d, J=8.0 Hz, 2H), 3.77 (t, J=5.7 Hz, 2H), 3.40 (t, J=5.8 Hz, 2H), 3.02 (s, 6H) 2.46 (s, 3H), and 1.86 (s, 9H); m/z calcd. for C23H30N6O2: 422.53. found: 423.30. HPLC retention time 3.138 min (gradient of solvent B—0 to 100%; wavelength 220 nM).

### 5.8. Preparation of 7-tert-Butyl-2-(4-methyl-benzoylamino)-7H-pyrrolo[2,3-d]pyrimidine-5-carboxylic acid methylamide

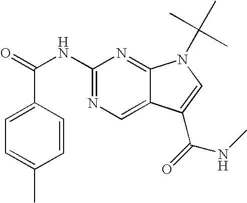

Under a blanket of nitrogen and in a scintillating vial, amide 11 (35 mg, 0.081 mmol) was dissolved in 1 ml of DMF. The solution was degassed using nitrogen and then trans-dichlorobis(triphenylphosphine)palladium (5.6 mg, 0.0081 mmol) was added. After degassing with nitrogen once more the mixture was bubbled through with carbon monoxide for 3 min. A 2 M solution of methylamine in THF (0.08 ml, 0.162 mmol) was added to the mixture and the vial was sealed. The mixture was stirred at 80° C. After stirring for 12 h the mixture was diluted with EtOAc and filtered through Celite. The filtrate was concentrated and the residue was purified by prep-HPLC to yield the title compound (23 mg, 77%) as a white solid: ¹H NMR (300 MHz, MeOD) δ 9.35 (s, 1H), 8.48 (s, 1H), 8.00 (d, J=8.6 Hz, 2H), 7.42 (d, J=7.2 Hz, 2H), 2.94 (s, 3H), 2.47 (s, 3H), and 1.88 (s, 9H); m/z calcd. for C20H23N5O2: 365.44. found: 366.25. HPLC retention time=3.443 min (gradient of solvent B—0 to 100%; wavelength 220 nM).

### 5.9. Preparation of 6-Amino-1-tert-butyl-1H-pyrrolo[2,3-b]pyridine-3-carbonitrile

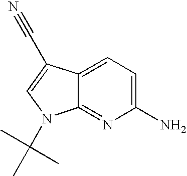

5-Amino-1-tert-butyl-1H-pyrrole-3-carbonitrile (15): To the sodium derivative of formyl-succinonitrile (14) (A. Brodrick and D. G. Wibberley, J. C. S. Perkin I, 1975, 1911) (1.0 g, 7.7 mmol) dissolved in ethanol was added 2 ml of acetic acid and then tert-butyl amine (0.85 ml, 8.1 mmol). The solution was stirred at reflux. After 45 min the mixture was cooled to room temperature. To the stirring solution was added a solution of KOH (2.68 g, 47.7 mmol) in ethanol. The resulting mixture was stirred again at reflux. After 45 min the reaction was cooled to room temperature and the solvent was removed with the rotary evaporator. The residue was diluted with water and EtOAc. The layers were partitioned and the aqueous layer was extracted twice more with EtOAc. The organic phases were combined, dried over MgSO₄ and concentrated to yield the pyrrole 15 (791 mg, 63%): ¹H NMR (300 MHz, (MeOD) δ 7.11 (d, J=2.3 Hz, 1H), 5.67 (d, J=2.2 Hz, 1H), 1.61 (s, 9H); m/z calcd. for C9H13N3: 163.22. found: 163.95. HPLC retention time=1.550 min (Column: Luna C8 4.6×50 mm, Gradient time: 3 min, flow rate: 2 mmin, gradient of solvent B—0 to 100%; wavelength 220 nM).

6-Amino-1-tert-butyl-1H-pyrrolo[2,3-b]pyridine-3-carbonitrile (17): To a solution of pyrrole 4 (500 mg, 3.05 mmol) in 50 ml of EtOH was added 3,3-dimethoxypropionitrile (16) (350 mg, 3.05 mmol) and then 1 ml of concentrated hydrochloric acid. The solution was stirred at reflux. After 2 h the solvent was removed with the rotary evaporator. The residue was diluted with water and then neutralized with 1 N NaOH_{(aq)}. The aqueous mixture was extracted with EtOAc. The organic layer was separated, dried over MgSO₄ and concentrated. The crude product was purified by silica gel column chromatography to yield the pyrrolopyridine 17 (607 mg, 93%): ¹H NMR (400 MHz, (CDCl₃) δ 7.93 (d, J=8.8 Hz, 1H), 7.57 (s, 1H), 6.60 (d, J=8.8 Hz, 1H), 1.76 (s, 9H); m/z calcd. for C12H14N4: 214.27. found: 214.90. HPLC retention time=3.395 min (Column: ShimPack VP-ODS 50×4.6, Gradient time: 4 min, flow rate: 2.5 ml/min, gradient of solvent B—0 to 100%; wavelength 220 nM).

### 5.10. Preparation of 1-tert-Butyl-6-(4-methyl-benzoylamino)-1H-pyrrolo[2,3-b]pyridine-3-carboxylic acid ethyl ester

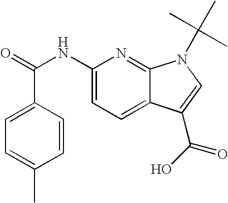

6-Amino-1-tert-butyl-1H-pyrrolo[2,3-b]pyridine-3-carboxylic acid ethyl ester (18): To a solution of the pyrrolopyridine 17 (150 mg, 0.70 mmol) in 20 ml of EtOH was added 5 ml of sulfuric acid. The solution was stirred at reflux overnight. The solvent was then removed in vacuo. The residue was diluted with water and then neutralized with 1 N NaOH_{(aq)}. The aqueous mixture was extracted with EtOAc. The organic layer was separated, dried over MgSO₄ and concentrated. The crude product was purified by prep-HPLC to yield the ester 18: ¹H NMR (400 MHz, (CDCl₃) δ 9.35 (s, 2H), 8.44 (d, J=8.8 Hz, 1H), 7.71 (s, 1H), 6.72 (d, J=8.8 Hz, 1H), 4.36 (q, J=7.2 Hz, 2H), 1.76 (s, 9H), 1.39 (t, J=7.2 Hz, 3H); m/z calcd. for C14H19N3O2: 261.33. found: 261.95. HPLC retention time=3.625 min (Column: ShimPack VP-ODS 50×4.6, Gradient time: 4 min, flow rate: 2.5 ml/min, gradient of solvent B—0 to 100%; wavelength 220 nM).

1-tert-Butyl-6-(4-methyl-benzoylamino)-1H-pyrrolo[2,3-b]pyridine-3-carboxylic acid ethyl ester (20): To a solution of ester 7 (1.2 g, 5.6 mmol) in pyridine was added p-toluoyl chloride (19) (1.02 ml, 11.2 mmol). The reaction was stirred at room temperature. After stirring for 2 h the solvent was removed with the rotary evaporator. The residue was diluted with EtOAc and then washed with brine. The organic layer was dried over MgSO₄ and concentrated. The crude product was purified by prep-HPLC to yield the amide 20 (1.37 g, 65%): ¹H NMR (400 MHz, (CDCl₃) δ 8.45 (d, J=8.8 Hz, 1H), 8.22 (d, J=8.8 Hz, 1H), 8.00 (s, 1H), 7.86 (d, J=8.0 Hz, 2H), 7.28 (d, J=8.0 Hz, 2H), 4.36 (q, J=7.2 Hz, 2H), 2.40 (s, 3H), 1.80 (s, 9H), 1.41 (t, J=7.6 Hz, 3H); m/z calcd. for C22H25N3O3: 379.46. found: 379.95. HPLC retention time=4.590 min (Column: ShimPack VP-ODS 50×4.6, Gradient time: 4 min, flow rate: 3.0 ml/min, gradient of solvent B—0 to 100%; wavelength 220 nM).

1-tert-Butyl-6-(4-methyl-benzoylamino)-1H-pyrrolo[2,3-b]pyridine-3-carboxylic acid (21): To a solution of the ester 20 (83 mg, 0.218 mmol) in ethanol was added 4 ml of 1 N NaOH_{(aq)}. The mixture was stirred at 70° C. overnight. The mixture was then diluted with EtOAc and the layers were separated. The aqueous layer was acidified with 1 N HCl_{(aq)}. The precipitate was filtered to give the desired acid 21: m/z calcd. for C20H21N3O3: 351.41. found: 351.95.

### 5.11. Preparation of 1-tert-Butyl-6-(4-methyl-benzoylamino)-1H-pyrrolo[2,3-b]pyridine-3-carboxylic acid isopropylamide

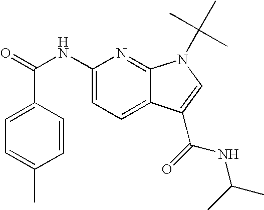

To a solution of the acid 21 (30 mg, 0.08 mmol) in DMF was added isopropylamine (0.015 ml, 0.17 mmol), then N,N,N′,N′-Tetramethyl-O-(7-azabenzotriazol-1-yl)uronium hexafluorophosphate (65 mg, 0.17 mmol) and then triethylamine (0.023 ml, 0.17 mmol). The solution was stirred at room temperature. After 12 h the mixture was concentrated. The residue was purified by prep-HPLC to yield the title compound (3.4 mg, 11%): 1H NMR (400 MHz, (CDCl3) δ 8.53 (bs, !H), 8.29 (d, J=8.8 Hz, 1H), 8.17 (d, J=8.8 Hz, 1H), 7.94 (s, 1H), 7.86 (d, J=8.0 Hz, 2H), 7.33 (d, J=8.0 Hz, 2H), 5.88 (bs, 1H), 4.39-4.32 (m, 1H), 2.45 (s, 3H), 1.79 (s, 9H), 1.32 (d, J=6.8 Hz, 6H); m/z calcd. for C23H28N4O2: 392.51. found: 393.00. HPLC retention time=4.193 min (Column: ShimPack VP-ODS 50×4.6, Gradient time: 4 min, flow rate: 3.0 ml/min, gradient of solvent B—15 to 100%; wavelength 220 nM).

### 5.12. Preparation of 1-tert-Butyl-6-(4-methyl-benzoylamino)-1H-pyrrolo[2,3-b]pyridine-3-carboxylic acid ethylamide

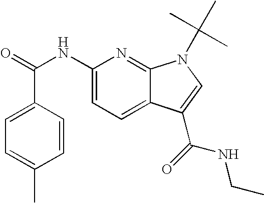

To a solution of the acid 21 (50 mg, 0.14 mmol) in DMF was added ethylamine (0.140 ml, 0.28 mmol), then N,N,N′,N′-Tetramethyl-O-(7-azabenzotriazol-1-yl)uronium hexafluorophosphate (108 mg, 0.28 mmol) and then triethylamine (0.041 ml, 0.28 mmol). The solution was stirred at room temperature. After 12 h the mixture is concentrated. The residue is purified by prep-HPLC to yield the title compound (17 mg, 32%): ¹H NMR (400 MHz, (CDCl₃) δ 8.72 (bs, 1H), 8.21 (dd, J=8.8, 6.8 Hz, 2H), 7.94 (s, 1H), 7.85 J=8.0 Hz, 2H), 7.30 (d,J=8.4 Hz, 2H), 6.36 (bs, 1H), 3.51 (q, J=7.2 Hz, 2H), 2.43 (s, 3H), 1.76 (s, 9H), 1.26 (t, J=7.2 Hz, 3H); m/z calcd. for C22H26N4O2: 378.48. found: 379.00. HPLC retention time=5.168 min (Column: ShimPack VP-ODS 50×4.6, Gradient time: 5 min, flow rate: 3.0 ml/min, gradient of solvent B—10 to 100%; wavelength 220 nM).

### 5.13. Preparation of 1-tert-Butyl-6-(4-methyl-benzoylamino)-1H-pyrrolo[2,3-b]pyridine-3-carboxylic acid isobutyl-amide

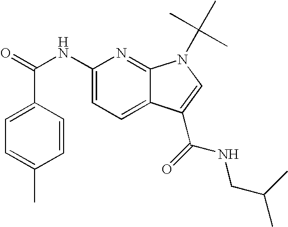

To a solution of the acid 21 (50 mg, 0.14 mmol) in DMF was added isobutylamine (0.028 ml, 0.28 mmol), then N,N,N′,N′-Tetramethyl-O-(7-azabenzotriazol-1-yl)uronium hexafluorophosphate (108 mg, 0.28 mmol) and then triethylamine (0.041 ml, 0.28 mmol). The solution was stirred at room temperature. After 12 h the mixture is concentrated. The residue is purified by prep-HPLC to yield the title compound (22 mg, 24%): ¹H NMR (400 MHz, (CDCl₃) δ 8.60 (bs, 1H), 8.27 (d, J=8.8 Hz, 1H), 8.17 (d, J=8.4 Hz, 1H) 7.97 (s, 1H), 7.86 (d, J=8.4 Hz, 2H), 7.33 (d, J=7.6 Hz, 2H), 6.20 (bs, 1H), 3.34 (d, J=6.8 Hz, 2H), 2.45 (s, 3H), 1.99-1.90 (m, 1H), 1.76 (s, 9H), 1.01 (d, J=6.8 Hz, 3H); m/z calcd. for C24H30N4O2: 406.53. found: 407.05. HPLC retention time=4.796 min (Column: ShimPack VP-ODS 50×4.6, Gradient time: 5 min, flow rate: 3.0 ml/min, gradient of solvent B—30 to 100%; wavelength 220 nM).

### 5.14. Preparation of 1-tert-Butyl-6-(4-methyl-benzoylamino)-1H-pyrrolo[2,3-b]pyridine-3-carboxylic acid dimethylamide

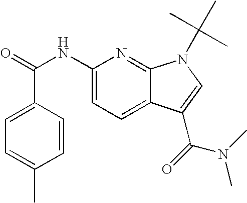

To a solution of the acid 21 (50 mg, 0.14 mmol) in DMF was added dimethylamineamine (0.140 ml, 0.28 mmol), then N,N,N′,N′-Tetramethyl-O-(7-azabenzotriazol-1-yl)uronium hexafluorophosphate (108 mg, 0.28 mmol) and then triethylamine (0.041 ml, 0.28 mmol). The solution was stirred at room temperature. After 12 h the mixture is concentrated. The residue is purified by prep-HPLC to yield the title compound (5.3 mg, 10%): ¹H NMR (400 MHz, (CDCl₃) δ 8.42 (bs, 1H), 8.26 (d, J=8.8 Hz, 1H), 8.06 (d, J=9.6 Hz, 1H), 7.86 (d, J=7.2 Hz, 2H), 7.64 (s, 1H), 7.32 (d, J=7.6 Hz, 2H), 3.17 (s, 6H), 2.44 (s, 3H), 1.79 (s, 9H); m/z calcd. for C22H26N4O2: 378.48. found: 379.00. HPLC retention time=3.455 min (Column: ShimPack VP-ODS 50×4.6, Gradient time: 4 min, flow rate: 3.0 ml/min, gradient of solvent B—40 to 100%; wavelength 220 nM).

### 5.15. Preparation of 1-tert-Butyl-6-(4-methyl-benzoylamino)-1H-pyrrolo[2,3-b]pyridine-3-carboxylic acid diethylamide

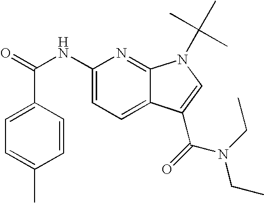

To a solution of the acid 21 (50 mg, 0.14 mmol) in DMF was added diethylamine (0.05 ml, 0.28 mmol), then N,N,N′,N′-tetramethyl-O-(7-azabenzotriazol-1-yl)uronium hexafluorophosphate (108 mg, 0.28 mmol) and then triethylamine (0.041 ml, 0.28 mmol). The solution was stirred at room temperature. After 12 hours the mixture is concentrated. The residue is purified by prep-HPLC to yield the title compound (9.9 mg, 17%): ¹H NMR (400 MHz, (CDCl₃) δ 8.49 (bs, 1H), 8.24 (d, J=8.8 Hz, 1H), 8.00 (d, J=8.8 Hz, 1H), 7.86 (d, J=8.0 Hz, 2H), 7.65 (s, 1H), 7.33 (d, J=8.4 Hz, 2H), 3.61 (q, J=7.2 Hz, 4H), 2.45 (s, 3H), 1.79 (s, 9H), 1.26 (t, J=7.2 Hz, 6H); m/z calcd. for C24H30N4O2: 406.53. found: 407.00. HPLC retention time=4.545 min (Column: ShimPack VP-ODS 50×4.6, Gradient time: 5 min, flow rate: 3.0 ml/min, gradient of solvent B—30 to 100%; wavelength 220 nM).

### 5.16. Preparation of 1-tert-Butyl-6-fluoro-1H-pyrazolo[3,4-b]pyridine-3-carboxylic acid

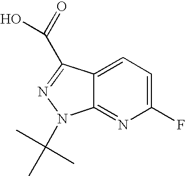

(2,6-Difluoro-pyridin-3-yl)-oxo-acetic acid tert-butyl ester (23): To a solution of 2,6-difluoropyridine (22) (2.7 ml, 30 mmol) in 30 ml of THF at −78° C. was added dropwise a freshly prepared solution of lithium diisopropylamine (32 mmol). The resulting solution was maintained at −78° C. for 30 min. To the stirring solution was added dropwise a preloaded solution of di-tert-butyl oxylate (7.7 g, 38 mmol) in 30 ml of THF at −78° C. The reaction mixture was stirred at −78° C. for 30 min and then at −20° C. for 20 min. The solution was quenched with a saturated solution of NH4Cl_{(aq)} and then diluted with Et₂O. The layers were separated and the organic layer was dried over Na₂SO₄ and then concentrated in vacuo to yield the product 23 (6.93 g, 95%) as a yellow oil: ¹H NMR (300 MHz, (CDCl₃) δ 8.49 (dd, 1H), 7.04 (dd, J_{HH}=8.2 Hz, J_{HF}=2.9 Hz, 1H), 1.61 (s, 9H).

(tert-Butyl-hydrazono)-(2,6-difluoro-pyridin-3-yl)-acetic acid tert-butyl ester (24): To a solution of the difluoropyridine 23 (8.0 g, 32.9 mmol) in EtOH was added tert-butylhydrazine (4.1 g, 32.9 mmol) and triethylamine (4.58 ml, 32.9 mmol). The reaction was stirred at 60° C. After stirring for 2 h the mixture was concentrated in vacuo. The residue was diluted with brine and methylene chloride. The layers were separated and the organic layer was dried over MgSO₄ and concentrated. The crude product was purified by silica gel column chromatography to yield the product 24 (2.25 g, 22%): ¹H NMR (400 MHz, (CDCl₃) 67 7.82 (dd, 1H), 7.04 (dd, J_{HH}=8.0 Hz, J_{HF}=3.0 Hz, 1H), 1.47 (s, 9H), 1.27 (s, 9H).

1-tert-Butyl-6-fluoro-1H-pyrazolo[3,4-b]pridine-3-carboxylic acid tert-butyl ester (25): To a solution of 24 (2.3 g, 7.35 mmol) in THF was added sodium hydride (340 mg, 8.81 mmol). The reaction was stirred at 70° C. and followed using TLC. Upon completion the mixture was quenched with a saturated solution of NH4Cl_{(aq)} and then diluted with brine. The layers were separated and the organic layer was dried over MgSO₄ and concentrated in vacuo. The crude product was purified by silica gel column chromatography to yield the ester 25 (1.2 g, 56%): ¹H NMR (300 MHz, (CDCl₃) δ 8.45 (dd, 1H), 6.90 (dd, J_{HH}=8.6 Hz, J_{HF}=1.4 Hz, 1H), 1.86 (s, 9H), 1.70 (s, 9H); m/z calcd. for C15H20FN3O2: 293.34. found: 293.90. HPLC retention time=3.726 min (Gradient time: 3 min, flow rate: 2.5 ml/min, gradient of solvent B—50 to 100%; wavelength 220 nM).

1-tert-Butyl-6-fluoro-1H-pyrazolo[3,4-b]pyridine-3-carboxylic acid (26): To a solution of the ester 25 (1.2 g, 4.1 mmol) in 40 ml of methylene chloride was added 5 ml of trifluoroacetic acid. After stirring for 4 h the mixture was concentrated to yield the acid 26.

5.17. Preparation of 1-tert-Butyl-6-(4-methyl-benzoylamino)-1H-pyrazolo[3,4-b]pyridine-3-carboxylic acid (1-ethyl-propyl)-amide

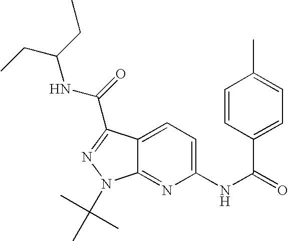

6-Amino-1-tert-butyl-1H-pyrazolo[3,4-b]pyridine-3-carboxylic acid (1-ethyl-propyl)-amide (27): To a solution of acid 26 (158 mg, 0.667 mmol), triethylamine (0.1 ml, 0.733 mmol), EDCI (140 mg, 0.733 mmol) and HOAt (100 mg, 0.733 mmol) in methylene chloride was added 1-ethylpropane (58 mg, 0.667 mmol). The mixture was stirred at room temperature overnight. The reaction was then washed with brine. The organic layer was separated, dried over MgSO₄ and concentrated to give a yellow oil. The crude intermediate was taken up in 10 ml of 7 N ammonia dissolved in methanol. The solution was stirred at 140° C. After 36 h the mixture was concentrated. The crude product was purified by prep-HPLC to yield the amide 27 (60 mg, 30%) as a clear oil: m/z calcd. for C16H25N5O: 303.41. found: 304.20.

1-tert-Butyl-6-(4-methyl-benzoylamino)-1H-pyrazolo[3,4-b]pyridine-3-carboxylic acid (1-ethyl-propyl)-amide (29): To a solution of amide 7 (80 mg, 0.264 mmol) in 3 ml of pyridine was addedp-toluoyl chloride (0.087 ml, 0.66 mmol). The reaction was stirred at room temperature and followed using TLC. After 4 h of stirring the solvent was removed with the rotary evaporator. The residue was diluted with methylene chloride and then washed with a saturated solution of NaHCO₃(aq) and brine, The organic layer was dried over MgSO₄ and concentrated. The crude product was purified by prep-HPLC to yield the title compound (57 mg, 51%) as a white solid: ¹H NMR (300 MHz, (CDCl₃) δ 8.71 (d, J=9.0 Hz, 1H), 8.52 (bs, 1H), 8.39 (d, J=8.7 Hz, 1H), 7.89 (d, J=8.4 Hz, 2H), 7.36 (d, J=7.8 Hz, 2H), 6.75 (d, J=9.5 Hz, 1H), 4.11-3.97 (m, 1H), 2.47 (s, 3H), 1.85 (s, 9H), 1.71-167 (m, 2H), 1.61-1.55 (m, 2H), 1.02 (t, J=7.2 Hz, 6H); m/z calcd. for C24H31N5O2: 421.55. found: 422.30. HPLC retention time=4.731 min (Gradient time: 3 min, flow rate: 3 ml/min, gradient of solvent B—40 to 100%; wavelength 220 nM).

### 5.18. Preparation of 1-tert-Butyl-6-(4-methyl-benzoylamino)-1H-pyrazolo[3,4-b]pyridine-3-carboxylic acid isopropylamide

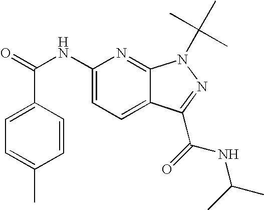

6-Amino-1-tert-butyl-1H-pyrazolo[3,4-b]pyridine-3-carboxylic acid isopropylamide (29): To a solution of acid 26 (200 mg, 0.844 mmol), triethylamine (0.142 ml, 1.02 mmol), EDCI (198 mg, 1.02 mmol) and HOAt (137 mg, 1.02 mmol) in 5 ml of methylene chloride was added isopropylamine (0.072 ml, 0.844 mmol). The mixture was stirred at room temperature overnight. The reaction was then washed with a saturated solution of NaHCO_{3(aq)} and brine. The organic layer was separated, dried over MgSO₄ and concentrated to give a yellow solid. The crude intermediate was taken up in 7 N ammonia dissolved in methanol. The solution was stirred at 140° C. After 24 h the mixture was concentrated. The crude product was purified by prep-HPLC to yield the amide 29 (99 mg, 43%) as a white solid: m/z calcd. for C14H21N5O: 275.36. found: 276.1.

1-tert-Butyl-6-(4-methyl-benzoylamino)-1H-pyrazolo[3,4-b]pyridine-3-carboxylic acid isopropylamide (30): To a solution of amide 29 (60 mg, 0.218 mmol) in 4 ml of pyridine was added p-toluoyl chloride (0.051 ml, 0.436 mmol). The reaction was stirred at room temperature. After 4 h of stirring the solvent was removed with the rotary evaporator. The residue was diluted with 50 ml of methylene chloride and then washed with a saturated solution of NaHCO_{3(aq)} and brine. The organic layer was dried over MgSO₄ and concentrated. The crude product was purified by prep-HPLC to yield the title compound (38 mg, 44%) as a white solid: ¹H NMR (300 MHz, (CDCl₃) δ 8.70 (d, J=8.7 Hz, 1H), 8.51 (bs, 1H), 8.38 (d, J=8.7 Hz, 1H), 7.89 (d, J=8.4 Hz, 2H), 7.36 (d, J=7.8 Hz, 2H), 6.84 (d, J=9.0 Hz, 1H), 4.42-4.29 (m, 1H), 2.48 (s, 3H), 1.85 (s, 9H), 1.34 (d, J=6.6, 6H); m/z calcd. for C22H27N5O2: 393.49. found: 394.30. HPLC retention time=4.371 min (Gradient time: 3 min, flow rate: 3 ml/min, gradient of solvent B—50 to 100%; wavelength 220 nM).

### 5.19. Preparation of 6-Amino-1-tert-butyl-1H-pyrazolo[3,4-b]pyridine-3-carboxylic acid cyclopropylamide

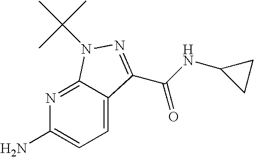

To a solution of acid 26 (150mg, 0.632mol), triethylamine (0.07 ml, 0.76 mmol), EDCI (145 mg, 0.76 mmol) and HOAt (103 mg, 0.76 mmol) in methylene chloride was added cyclopropylamine (36 mg, 0.632 mmol). The mixture was stirred at room temperature overnight. The reaction was then washed with a saturated solution of NaHCO_{3(aq)} and brine. The organic layer was separated, dried over MgSO₄ and concentrated. The crude intermediate was taken up in 7 N ammonia dissolved in methanol. The solution was stirred at 140° C. After 24 h the mixture was concentrated. The crude product was purified by prep-HPLC to yield the title amide (50 mg, 27%) as a white solid: m/z calcd. for C14H19N5O: 273.34. found: 274.2.

1-tert-Butyl-6-(4-methyl-benzoylamino)-1H-pyrazolo[3,4-b]pyridine-3-carboxylic acid cyclopropylamide: To a solution of 6-Amino-1-tert-butyl-1H-pyrazolo[3,4-b]pyridine-3-carboxylic acid cyclopropylamide (50 mg, 0.169 mmol) in 2 ml of pyridine was added p-toluoyl chloride (0.05 ml, 0.378 mmol). The reaction was stirred at room temperature overnight. The solvent was removed with the rotary evaporator. The residue was diluted with 50 ml of methylene chloride and then washed with a saturated solution of NaHCO_{3(aq)} and brine. The organic layer was dried over MgSO₄ and concentrated. The crude product was purified by prep-HPLC to yield the title compound (25 mg, 38%) as a white solid: ¹H NMR (300 MHz, (CDCl₃) δ 8.70 (d, J=9.0 Hz, 1H), 8.51 (bs, 1H), 8.40 (d, J=9.0 Hz, 1H), 7.89 (d, J=8.1 Hz, 2H), 7.36 (d, J=8.1 Hz, 2H), 7.11 (bs, 1H), 2.97-2.87 (m, 1H), 2.47 (s, 3H), 1.83 (s, 9H), 0.95-0.88 (m, 2H), 0.75-0.70 (m, 2H); m/z calcd. for C22H25N5O2: 391.48. found: 392.45. HPLC retention time=4.201 min (Gradient time: 3 min, flow rate: 3 ml/min, gradient of solvent B—50 to 100%; wavelength 220 nM).

### 5.20. Preparation of 1-tert-Butyl-6-(3-methyl-benzoylamino)-1H-pyrazolo[3,4-b]pyridine-3-carboxylic acid isopropylamide

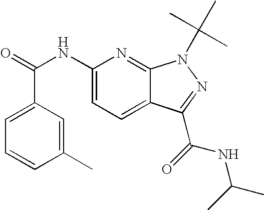

To a solution of m-toluoyl chloride (0.025 ml, 0.18 mmol) in 0.5 ml of pyridine was added a solution of the amide 29 (38 mg, 0.18 mmol) in 1.5 ml of pyridine. The resulting solution was stirred at room temperature for 3 h and then concentrated. The crude product was purified by prep-HPLC to yield the title compound as a white solid: ¹H NMR (300 MHz, (CDCl₃) δ 8.61 (d, J=8.7 Hz, 1H), 8.39 (s, 1H), 8.28 (d, J=9.0 Hz, 1H), 7.72-7.62 (m, 2H), 7.36-7.31 (m, 2H), 6.72 (d, J=7.8 Hz, 1H), 4.33-4.19 (m, 1H), 2.39 (s, 3H), 1.75 (s, 9H), 1.24 (d J=6.6, 6H); m/z calcd. for C22H27N5O2: 393.49. found: 394.35.

5.21. Human Proline Transporter Assay

The ability of compounds to inhibit the proline transporter was determined as follows. A human SLC6A7 cDNA was cloned into a pcDNA3.1 vector and transfected into COS-1 cells. A cell clone stably expressing proline transporter was selected for the assay.

Transfected cells were seeded at 15,000 cells per well in a 384 well plate and grown overnight. The cells were then washed with Krebs-Ringer's-HEPES-Tris (KRHT) buffer, pH 7.4, containing 120 mM NaCl, 4.7 mM KCl, 2.2 mM CaCl, 1.2 mM MgSO4, 1.2 mM KH₂PO₄, 10 mM HEPES and 5 mM Tris. The cells were then incubated with 50 μl of KRHT buffer containing 45 nM ³H-Proline for 20 minutes at room temperature. Radiolabeled proline uptake was terminated by removing the radiolabeled proline and washing the cells rapidly three times with 100 μl of ice-cold KRHT buffer. Scintillation fluid (50 μl) was added per well, and the amount of tritiated proline present was determined using a Packard TopCount Scintillation counter.

Nonspecific uptake was determined by measuring of ³H-proline uptake in the presence of 2 mM cold proline.

The IC₅₀ of a compound was determined by measuring inhibition of four separate samples at ten concentrations, typically beginning with 10 μM followed by nine three-fold dilutions (i.e., 10, 3.3, 1.1, 0.37, 0.12, 0.41, 0.014, 0.0046, 0.0015, and 0 μM). Percent inhibitions were calculated against the control. The IC₅₀ of a compound was determined using the ten data points, each of which was an average of the four corresponding measurements.

5.22. Murine Proline Transporter Assay

Forebrain tissue was dissected from a wild type mouse and homogenized in 7 ml ice-cold homogenization buffer: 0.32 M sucrose, 1 mM NaHCO₃, protease inhibitor cocktail (Roche).

The brain homogenates were centrifuged at 1000×g for 10 min to remove nuclei. Supernatant was collected and re-centrifuged at 20000×g for 20 min to pellet crude synaptosomes. The synaptosomes were resuspended in ice-cold assay buffer: 122 mM NaCl, 3.1 mM KCl, 25 mM HEPES, 0.4 mM KH₂PO₄, 1.2 mM MgSO₄, 1.3 mM CaCl₂, 10 mM dextrose at pH 7.4. Resuspended synaptosomes were centrifuged again at 20000×g for 20 minutes, and pelleted synaptosomes were resuspended in assay buffer. Protein concentration was measured by DC protein assay kit (BioRad).

Proline transport assay was performed in 100 μl reaction mix consisting of 10 μg synaptosomes, 1 μCi/0.24 μM [H3]-proline in assay buffer for a time between 0 to 20 minutes at room temperature. The reaction was terminated by rapid filtration through GF/B filter plate (Millipore) followed by three rapid washes in 200 ul ice-cold assay buffer. Fifty microliters of Microscint-20 was added to each reaction and incubated for 2 hours. The [H3]-proline transport was determined by radioactivity counting.

To determine proline transport inhibition by compounds, compounds were incubated with the reaction mixture at concentrations ranging from 0 to 10 μM (11 points, beginning at 10 um; 3-fold dilutions; 4 replicates averaged to provide one point). The baseline activity, or nonspecific activity, was measured in the presence of 0.3 mM GGFL (Enkephalin, Sigma) in the reaction. The nonspecific activity was also measured in synaptosomes of SLC6A7 knockout mice. The nonspecific activities measured by the two methods were found to be identical.

5.23. Human Dopamine Transporter Assay

The ability of compounds to inhibit the dopamine transporter was determined as follows. A human DAT cDNA (NM_{—}001044) was cloned into a pcDNA3.1 vector and transfected into COS-1 cells. The resulting cell lines that stably express the dopamine transporter were used for further experimentation.

Transfected cells were seeded at 15,000 cells per well in a 384 well plate and grown overnight. The cells were then washed with Krebs-Ringer's-HEPES-Tris (KRHT) buffer, pH 7.4, containing 125 mM NaCl, 4.8 mM KCl, 1.3 mM CaCl₂, 1.2 mM MgSO₄ 10 mM D-glucose, 25 mM HEPES, 1 mM sodium ascorbate and 1.2 mM KH₂PO₄. The cells were then incubated with 50 μl of KRHT buffer containing 1 μM ³H-Dopamine for 10 minutes at room temperature. Radiolabeled dopamine uptake was terminated by removing the radiolabeled dopamine and washing the cells rapidly three times with 100 μl of ice-cold KRHT buffer. Scintillation fluid (50 μl) was added per well and the amount of tritiated dopamine present was determined using a Packard TopCount Scintillation counter.

Nonspecific uptake was determined by measuring of ³H-dopamine uptake in the presence of 250 μM benztropine. The IC₅₀ of a compound was determined by measuring inhibition of four separate samples at ten concentrations, typically beginning with 10 μM followed by nine three-fold dilutions (i.e., 10, 3.3, 1.1, 0.37, 0.12, 0.41, 0.014, 0.0046, 0.0015, and 0 μM). Percent inhibitions were calculated against the control. The percentage inhibitions were calculated against the control, and the average of the quadruplicates was used for IC₅₀ calculation.

5.24. Human Glycine Transporter Assay

The ability of compounds to inhibit the glycine transporter was determined as follows. A human glycine transporter cDNA (NM_{—}006934) was cloned into a pcDNA3.1 vector and transfected into COS-1 cells. The resulting cell lines that stably express the glycine transporter were used for further experimentation.

Transfected cells were seeded at 15,000 cells per well in a 384 well plate and grown overnight. The cells were then washed with Krebs-Ringer's-HEPES-Tris (KRHT) buffer, pH 7.4, containing 120 mM NaCl, 4.7 mM KCl, 2.2 mM CaCl₂, 1.2 mM MgSO₄, 1.2 mM KH₂PO₄, 10 mM HEPES and 5 mM Tris. The cells were then incubated with 50 μl of KRHT buffer containing 166 nM ³H-glycine for 10 minutes at room temperature. Radiolabeled glycine uptake was terminated by removing the radiolabeled glycine and washing the cells rapidly three times with 100 μl of ice-cold KRHT buffer. Scintillation fluid (50 μl) was added per well and the amount of tritiated glycine present was determined using a Packard TopCount Scintillation counter.

Nonspecific uptake was determined by measuring ³H-glycine uptake in the presence of 2 mM cold glycine. The IC₅₀ of a compound was determined by measuring inhibition of four separate samples at ten concentrations, typically beginning with 10 μM followed by nine three-fold dilutions (i.e., 10, 3.3, 1.1, 0.37, 0.12, 0.41, 0.014, 0.0046, 0.0015, and 0 μM). Percent inhibitions were calculated against the control. The percentage inhibitions were calculated against the control, and the average of the quadruplicates was used for IC₅₀ calculation.

5.25. Calculating IC₅₀ Values

The IC₅₀ of a compound with regard to a given target is determined by fitting the relevant data, using the Levenburg Marquardt algorithm, to the equation:
y=A+((B−A)/(1+((C/x)ˆD)))
wherein A is the minimum y value; B is the maximum y value; C is the IC₅₀; and D is the slope. The calculation of the IC₅₀ is performed using XLFit4 software (ID Business Solutions Inc., Bridgewater, N.J. 08807) for Microsoft Excel (the above equation is model 205 of that software).

1. (canceled)

2. A compound of formula I: 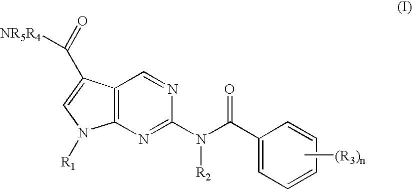
or a pharmaceutically acceptable salt or solvate thereof, wherein:
R₁ is hydrogen or optionally substituted alkyl, aryl, heterocycle, alkyl-aryl or alkyl-heterocycle;
R₂ is hydrogen or optionally substituted alkyl;
each R₃ is independently halogen, amine, hydroxy, alkoxy, or optionally substituted alkyl, aryl or heterocycle;
R₄ and R₅ are each independently hydrogen or optionally substituted alkyl, aryl, heterocycle, alkyl-aryl or alkyl-heterocycle, or taken together with the nitrogen atom to which they are attached, form an optionally substituted heterocycle; and
n is 0 to 5,
with the proviso that R₄ and R₅ together with the nitrogen atom to which they are attached do not form 1,4-diaza-bicyclo[3.2.2]nonane or piperazine-C(O)-phenyl.

3. The compound of claim 2, wherein R₁ is t-butyl or propyl.

4. The compound of claim 2, wherein R₃ is lower alkyl.

5. The compound of claim 2, wherein R₄ and R₅ are taken together to form optionally substituted pyridine or pyrrolidine.

6. (canceled)

7. (canceled)

8. (canceled)

9. (canceled)

10. A compound of formula II: 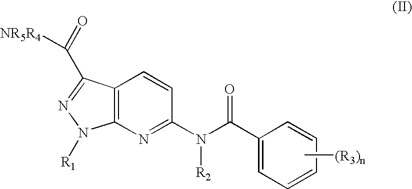
or a pharmaceutically acceptable salt or solvate thereof, wherein:
R₁ is hydrogen or optionally substituted alkyl, aryl, heterocycle, alkyl-aryl or alkyl-heterocycle;
R₂ is hydrogen or optionally substituted alkyl;
each R₃ is independently halogen, amine, hydroxy, alkoxy, or optionally substituted alkyl, aryl or heterocycle;
R₄ and R₅ are each independently hydrogen, or optionally substituted alkyl, aryl, heterocycle, alkyl-aryl or alkyl-heterocycle, or taken together with the nitrogen atom to which they are attached, form an optionally substituted heterocycle; and
n is 0 to 5.

11. The compound of claim 10, wherein R₁ is t-butyl or propyl.

12. The compound of claim 10, wherein R₃ is lower alkyl.

13. The compound of claim 10, wherein R₄ and R₅ are taken together to form optionally substituted pyridine or pyrrolidine.

14. (canceled)

15. (canceled)

16. (canceled)

17. (canceled)

18. (canceled)

19. A compound of formula III: 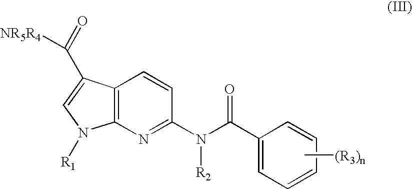
or a pharmaceutically acceptable salt or solvate thereof, wherein:
R₁ is hydrogen or optionally substituted alkyl, aryl, heterocycle, alkyl-aryl or alkyl-heterocycle;
R₂ is hydrogen or optionally substituted alkyl;
each R₃ is independently halogen, amine, hydroxy, alkoxy, or optionally substituted alkyl, aryl or heterocycle;
R₄ and R₅ are each independently hydrogen, or optionally substituted alkyl, aryl, heterocycle, alkyl-aryl or alkyl-heterocycle, or taken together with the nitrogen atom to which they are attached, form an optionally substituted heterocycle; and
n is 0 to 5.

20. The compound of claim 19, wherein R₁ is t-butyl or propyl.

21. The compound of claim 19, wherein R₃ is lower alkyl.

22. The compound of claim 19, wherein R₄ and R₅ are taken together to form optionally substituted pyridine or pyrrolidine.

23. The compound of claim 2, 10 or 19, which is a specific proline transporter inhibitor.

24. (canceled)

25. (canceled)

26. (canceled)

27. (canceled)

28. (canceled)

29. A pharmaceutical composition comprising a compound of claim 2, 10, or 19 and a pharmaceutically acceptable excipient.

30. (canceled)

31. (canceled)

32. A method of inhibiting a proline transporter, which comprises contacting a proline transporter with sufficient amount of a compound of claim 2, 10, or 19.

33. The method of claim 32, wherein the proline transporter is encoded by the human gene SLC6A7.

34-43. (canceled)
